(19) United States
(12) Patent Application Publication
(10) Pub. No.: US 2026/0301859 A1
(43) Pub. Date: Oct. 1, 2026

(54) COMPOSITIONS AND METHODS FOR MOLECULAR MIMICRY

(71) Applicant: Flagship Pioneering Innovations VII, LLC, Cambridge, MA (US)

(72) Inventors: Daniel William Menon ACKER, Arlington, MA (US); Jennifer Lai REMMEL, Jamaica Plain, MA (US); Jacob SIEGEL, Concord, MA (US)

(21) Appl. No.: 19/474,749

(22) PCT Filed: Apr. 12, 2024
(86) PCT No.: PCT/US24/24265

§ 371 Date: Oct. 13, 2025

Related U.S. Application Data
(60) Provisional application No. 63/459,333, filed on Apr. 14, 2023.
(60) Provisional application No. 63/459,332, filed on Apr. 14, 2023.

(51) Int. Cl. G16B 15/30; C40B 30/04; G01N 33/53; G01N 33/68; G16B 35/20

(52) U.S. Cl. CPC G16B 15/30; C40B 30/04; G01N 33/5308; G01N 33/6854; G01N 33/6863; G16B 35/20

(57) ABSTRACT

The present application is directed to methods for identifying and characterizing molecules that mimic binding sites of specific binders on various binding surfaces.

### FIELD OF THE DISCLOSURE

The present application is directed to methods for identifying and characterizing molecules that mimic binding sites of specific binders on various binding surfaces.

### CROSS-REFERENCE TO RELATED APPLICATIONS

This application claims the benefit of and priority to U.S. Provisional Patent Application Nos. 63/459,332, filed Apr. 14, 2023, and 63/459,333, filed Apr. 14, 2023, the contents of which are hereby incorporated by reference in their entireties.

### BACKGROUND

Limitations of therapeutic ligands, such as polypeptides, including immunoglobulins and related molecules, as therapeutic and diagnostic detection agents for various diseases and/or disorders have led to a push for designing an alternative class of molecular binding reagents-the mimetic molecule. Mimetic molecules are polymers that interact with a binding partner in a way that recapitulates the network of interactions used by another ligand. By mimicking the structural and/or energetic interactions at a ligand/receptor interface, the functional properties of the ligand can be recapitulated. For example, an antibody mimetic is a molecule that mimics the antigen binding activity of a specific antibody but is not identical (and in some embodiments, not related, e.g., exhibits no significant sequence similarity) in peptide sequence to that specific antibody or its paratope.

There are numerous applications of mimetic proteins ranging from utility as drug design and discovery tools to therapeutics and vaccines. To date, there has been no reliable and scalable method for identifying mimetic proteins.

### SUMMARY

Accordingly, in aspects, the present disclosure relates to the process of discovery of multiple classes of mimetic molecules that can be used as therapeutics, therapeutic precursors, and/or novel reagents/tool compounds, e.g., useful for screening interaction partners.

In aspects, the present disclosure provides methods of identifying a functional mimic molecule having a target functional profile that comprises one or more functions of a model by (a) providing one or more variant libraries of one or more functional mold molecules of the model, wherein the variant library comprises a plurality of variants of the one or more functional mold molecules, (b) providing one or more candidate functional mimic molecules, (c) evaluating the interaction of the plurality of variant functional mold molecules with (i) the model and (ii) the one or more candidate functional mimic molecules, and comparing the evaluations of i) and ii), and (d) identifying one or more functional mimic molecules having the target functional profile on the basis of the evaluation in c).

In aspects, the present disclosure provides methods of identifying a functional mimic molecule having a target functional profile that comprises one or more functions of a model by (a) providing one or more functional mold molecules of the model, (b) providing one or more variant libraries of one or more candidate functional mimic molecules, the variant libraries comprising a plurality of variants of the one or more candidate functional mimic molecules, (c) evaluating the interaction of the one or more functional mold molecules with: i) the model; and ii) the plurality of variants of the one or more candidate functional mimic molecules, and comparing the evaluations of i) and ii), and (d) identifying one or more functional mimic molecules having the target functional profile on the basis of the evaluation in c).

In aspects, the present disclosure provides methods of identifying a functional mimic molecule having a target functional profile that comprises one or more functions of a model by (a) providing one or more variant libraries of one or more functional mold molecules of the model, the variant libraries comprising a plurality of variants of the one or more functional mold molecules, (b) providing one or more variant libraries of one or more candidate functional mimic molecules, the variant libraries comprising a plurality of variants of the one or more candidate functional mimic molecules, (c) evaluating and comparing the interactions of the plurality of variant functional mold molecules with (i) the model and (ii) the plurality of variants of the one or more candidate functional mimic molecules, and (d) identifying one or more functional mimic molecules having the target functional profile on the basis of the evaluation in c).

The present disclosure further provides, in aspects, methods of identifying functional mimic molecules having target functional profiles that comprise one or more functions of a model by (a) evaluating and comparing the interaction of a plurality of variant functional mold molecules with (i) the model and (ii) one or more candidate functional mimic molecules, or a plurality of variant candidate functional mimic molecules thereof, and (b) identifying one or more functional mimic molecules having the target functional profile on the basis of the evaluation in (a).

In embodiments, the identified mimic molecule exhibits a non-zero similarity to the functional profile of the model.

In embodiments, the candidate functional mimic molecule is an immunoglobulin antibody, an antibody-like molecule, or an antigen-binding fragment thereof. In embodiments, the antibody or antibody-like molecule is selected from a bi-specific antibody, a tri-specific antibody, and an antibody-drug conjugate. In embodiments, the antigen-binding fragment is selected from a single-domain antibody (sdAb), a variable domain of heavy-chain antibodies (VHH), a single-chain antibody (scFv), a shark heavy-chain-only antibody (VNAR), a Fv, a Fab, a Fab′, a F(ab′)₂, and a microprotein (cysteine knot protein, knottin).

In embodiments, the functional mimic molecule is a functional mimic polypeptide that is an anti-idiotypic antibody, or fragment thereof, that binds the antigen-binding domain of the functional mold molecule or polypeptide, optionally the paratope.

In embodiments, the candidate functional mimic molecule is a small molecule (e.g., a molecular glue). In embodiments, the candidate functional mimic molecule is a small molecule (e.g., molecular glue) conjugated (e.g., covalently or non-covalently) to another domain (e.g., cereblon, E3-ligase, or immunomodulatory drugs). In embodiments, the molecular glue is conjugated to another domain via click chemistry (e.g., a reaction selected from one or more of conjugate addition, strained ring opening, acylation/sulfonylation, aldehyde capture by a-effect nucleophiles, cycloaddition, and nucleophilic addition to activated alkynes).

In embodiments, the candidate functional mimic molecule is a polymer, optionally selected from a polypeptide and a nucleic acid. In embodiments, the polymer is a nucleic acid that comprises DNA, RNA, or a combination thereof. In embodiments, the nucleic acid is selected from a DNA aptamer, a DNA optimer, a RNA aptamer, and a RNA optimer, any of which optionally comprise one or more non-canonical and/or modified nucleotides, optionally wherein the aptamer and/or optimer comprises an altered backbone. In embodiments, the nucleic acid is a ribozyme and/or riboswitch.

In embodiments, the polymer is a polypeptide, and the polypeptide optionally comprises one or more non-canonical amino acids, such as L-isomers of canonical or non-canonical amino acids (L-amino acids) and/or D-isomers of canonical or non-canonical amino acids (D-amino acids), e.g., 1, 2, 3, 4, 5, 6, 7, 8, 9, 10, or more non-canonical amino acids, such as about: 10, 20, 30, 40, 50, 60, 70, 80, 90, or 100% non-canonical amino acids.

In embodiments, the polypeptide is a binding agent. In embodiments, the binding agent is an immunoglobulin antibody, an antibody-like molecule, or an antigen-binding fragment thereof. In embodiments, the antibody or antibody-like molecule is selected from a bi-specific antibody, a tri-specific antibody, and an antibody-drug conjugate. In embodiments, the antigen-binding fragment is selected from a single-domain antibody (sdAb), a variable domain of heavy-chain antibodies (VHH), a single-chain antibody (scFv), a shark heavy-chain-only antibody (VNAR), a Fv, a Fab, a Fab′, a F(ab′)₂, and a microprotein (cysteine knot protein, knottin).

In embodiments, the binding agent is a non-immunoglobulin-based binding domain, optionally selected from an affimer, an affilin, an affitin, an affibody, an alphabody, an anticalin, an avimer, a DARPin, a fynomer, a gastrobody, a Kunitz domain, a monobody, a nanoCLAMP, a repebody, a pronectin, a centyrin, and an obody.

In embodiments, the non-immunoglobulin-based binding domain is an enzyme.

In embodiments, the non-immunoglobulin-based binding domain is a signaling protein, optionally wherein the signaling protein is selected from a cytokine, growth factor, or hormone.

In embodiments, the non-immunoglobulin-based binding domain is a structural protein such as a receptor.

In embodiments, the non-immunoglobulin-based binding domain is a ligand.

In embodiments, the non-immunoglobulin-based binding domain is an extracellular domain.

In embodiments, the non-immunoglobulin-based binding domain is a bacterial nucleic acid binding protein, such as a bacteriophage MS2 protein or a bacteriophage lambda N protein.

In embodiments, the binding agent is a bifunctional molecule, such as a fusion protein or non-fusion protein conjugate, optionally wherein the binding agent is conjugated to a carbohydrate (e.g., polysaccharide), a lipid, or a small molecule. In embodiments, the binding agent is glycosylated, lapidated, or otherwise conjugated.

In embodiments of the disclosure, the target functional profile comprises one or more functions of the mimic molecule or complex, relative to the model, optionally selected from one or more of the affinity, avidity, activity, specificity, and/or selectivity for a receptor or ligand; the molecular structure or surface features; and biological activity.

In embodiments of the disclosure, the model is a protein, a lipid, a carbohydrate, a nucleic acid, or an inorganic molecule, or a combination thereof. In embodiments, the model comprises an antigen, optionally selected from a viral protein, a bacterial protein, a protozoan protein, an Archean protein, a fungal protein, a mammalian protein, or an inorganic protein, a small molecule, a carbohydrate, a nucleic acid, a lipid, or a plastic. In embodiments, the model is a therapeutic molecule, such as a therapeutic antibody or antigen-binding fragment thereof, optionally wherein the antigen-binding fragment of the antibody is the paratope of the antibody.

In embodiments of the disclosure, the variant library comprises variants across the entire mold molecule or functional mimic molecule or a subset (e.g., a contiguous or a non-contiguous domain or set) of residue positions or moieties of the mold molecule or functional mimic molecule.

In embodiments, variant libraries of the present disclosure are systematically engineered, optionally by an alanine scan, a shotgun scan, or binomial mutagenesis.

In embodiments, variant libraries of the present disclosure are randomly engineered or semi-systematically engineered by random mutation or random mutation of specific set of residue positions.

In embodiments, the variant library is selected from a fixed amino acid substitution library, a subset of possible substitutions library, or a full-substitution library. In embodiments, the full substitution library comprises deletions and insertions.

In embodiments, the variant library is a deep mutational scan (DMS) library.

In embodiments, providing of one or more variant libraries of one or more functional mold molecules or functional mimic molecules further comprises generation of the one or more functional mold molecules or functional mimic molecules by a method selected from interactions reported in the scientific literature, phage display, immunization (e.g., of a mammal, such as a mouse, rat, rabbit, non-human primate, or human) with the model (optionally by isolating model-binding B-cells and performing BCR sequencing), obtaining model-binding B cells from a subject previously exposed (or suspected of being exposed) to the model, competitive-binding assays, depletion studies, and structure-based modeling (including de novo in silico molecule design).

In embodiments, the present disclosure provides functional mold molecules that exhibit a non-zero functional complementarity to a model or functional mimic molecule.

In embodiments, the functional mold molecule is a small molecule (e.g., a molecular glue). In embodiments, the functional mold molecule is a small molecule conjugated to another domain (e.g., cereblon, E3-ligase, imide drugs (IMiDs), e.g., thalidomide, pomalidomide, or lenalidomide). In embodiments, the molecular glue is conjugated to another domain via click chemistry (e.g., a reaction selected from one or more of conjugate addition, strained ring opening, acylation/sulfonylation, aldehyde capture by a-effect nucleophiles, cycloaddition, and nucleophilic addition to activated alkynes). In embodiments, the functional mold molecule is a molecular glue. In embodiments, a molecular glue is a type of small molecule stabilizer of protein-protein interactions that encourages two proteins to come together that normally wouldn't interact by changing the surface of their target proteins. In embodiments, molecular glue-induced protein proximity can either be stabilized/activated or destabilized/inactivated, which is dependent on the property of the small molecules and the involved proteins. In embodiments, a molecular glue comprises a molecule selected from one or more of plant hormones, auxin, jasmonate, immunomodulatory imide drugs (IMiDs) (e.g., CC-122, CC-220, CC-885, thalidomide, pomalidomide, and lenalidomide). In embodiments, a molecular glue comprises cereblon and/or E3-ligase. In embodiments, the functional mold molecule is a Proteolysis Targeting Chimera (PROTAC), which is a heterobifunctional molecule that forms a ternary complex with the target protein and E3-ligase by making two distinct small molecule-protein interactions.

In embodiments, the functional mold molecule is a polymer, such as a nucleic acid or a polypeptide, or a combination thereof.

In embodiments, the functional mold molecule is a nucleic acid that is selected from a DNA aptamer, a DNA optimer, a RNA aptamer, or a RNA optimer.

In embodiments, the functional mold molecule is a functional mold polypeptide comprising functional complementarity. In embodiments, the functional mold polypeptide binds the model.

In embodiments, the present disclosure provides for evaluating and comparing interactions based on binding affinity.

The details of one or more examples of the disclosure are set forth in the description below. Other features or advantages of the present disclosure will be apparent from the following drawings, detailed description of several examples, and also from the appended claims. The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### BRIEF DESCRIPTION OF THE DRAWINGS

FIG. 1 depicts candidate mimic binding to a commercial antibody Fab in a yeast surface display. The commercial antibody Fab was expressed on the surface of yeast and stained with a panel of anti-idiotypic antibodies discovered from a naïve human scFv library.

FIGS. 2A-D show commercial antibody WT and DMS library binding to the antigen. FIG. 2A depicts the presence of human kappa light chain, confirming the surface expression of wildtype commercial antibody (solid) and commercial antibody libraries (dashed and dotted) on yeast. FIG. 2B shows that after gating for Fab expression, the libraries (dashed and dotted lines) exhibited a greater phenotypic range of binding to antigen relative to WT (solid). FIGS. 2C-D show that both libraries with mutations of either heavy chain or mutations in the light chain exhibit antigen binding correlated with expression, except for those variants that appear to lose binding altogether.

FIGS. 3A-B show the validation of the commercial antibody DMS assay. FIG. 3A shows a scatterplot of variants from two replicate selections against antigen. Replicates are strongly correlated. FIG. 3B shows the mapping of sensitive sites, as identified by DMS to the paratope of the commercial antibody for antigen. The paratope is strongly enriched for sensitive sites.

FIGS. 4A-C show the result of scoring and clustering the anti-idiotypic antibodies for antigen mimicry. FIG. 4A shows the validation of the anti-idiotypic antibody DMS assays through a cumulative histogram of inter-replicate correlations. FIG. 4B shows the correlations of per variant binding scores from DMS between anti-idiotypic antibodies and antigen. The most highly correlated anti-idiotypic antibodies display similar correlations to a replicate antigen selection. FIG. 4C shows the result of clustering anti-idiotypic antibodies by DMS binding profiles. Clustering identifies groups of anti-idiotypic antibodies that are similar to the antigen, as well as more distant clusters.

FIGS. 5A-D are summaries showing the binding of individual DMS library variants to (A) antigen and (B-C) three anti-idiotypic antibodies. FIG. 5B shows an anti-idiotypic antibody that binds using a different combination of mold CDRs to antigen. FIG. 5C shows an anti-idiotypic antibody that binds using a similar set of CDRs but different residues. FIG. 5D shows an anti-idiotypic antibody that mimics antigen, as evinced through a similar set of sensitive sites indicating binding through the same set of residues. The Y axis denotes particular substitutions, and the X axis denotes the position along the peptide.

FIGS. 6A-C depict cryogenic electron microscopy structures of anti-idiotypic antibodies in complex with a commercial antibody. FIG. 6A shows an anti-drug antibody. FIG. 6B shows a discovered mimic anti-idiotypic antibody bound to a similar region. Overlapping structures, FIG. 6C, shows a highly similar epitope footprint on the commercial antibody despite minimal sequence similarity between the anti-idiotypic antibodies.

FIG. 7 shows the results of competitive inhibition ELISA measurements of the mold antibody in Example 2 in complex with a known mimic antibody. Increasing titrations of the model protein, unlabeled known mimic antibody and the discovered antibody show that all three are able to competitively displace the known mimic.

FIGS. 8A-C are summaries showing the binding of individual DMS variants to (A) a model protein, (B) a known mimic antibody, and (C) a discovered mimic antibody. FIG. 8B shows the known mimic antibody has similar sensitive sites to mutation as the model protein, with a notable exception of less sensitivity to mutations in the CDR1 region. FIG. 8C shows the discovered antibody binds similar to the known mimic antibody, but displays mutational sensitivity to binding more similar to the model protein in the CDR1 region. The Y axis denotes particular substitutions, and the X axis denotes the position along the peptide.

FIGS. 9A-D depict cryogenic electron microscopy structures of the broadly neutralizing antibody in complex with (A) the peptide epitope of the model protein, (B) the known discovered mimic antibody, and (C) the known mimic antibody. FIG. 9A shows the epitope forms a single loop structure that binds to a paratope made up of heavy chain CDR1, heavy chain CDR3, and light chain CDR3 of the broadly neutralizing antibody. FIG. 9B shows the CDR3 of the discovered antibody forms a similar loop structure to the epitope and binds the same paratope of the broadly neutralizing antibody mold. FIG. 9C shows the complex of the known mimic antibody bound to the broadly neutralizing antibody mold. While the paratope is similar, the epitope loop of known mimic adopts a dissimilar conformation. FIG. 9D displays the overlayed structures.

FIGS. 10A-D shows the competitive binding between the discovered antibodies in Example 3 and the model protein. FIG. 10A shows a reduction in binding of the model protein to an immobilized form of the extracellular domain of the receptor mold upon increasing concentrations of a discovered mimic antibody. FIG. 10B shows similar results for a second discovered mimic antibody. FIG. 10C shows the inhibition of mimic antibody in panel A binding to the receptor mold presented on HEK-293 cells by co-expression of a surface bound form of the model protein. FIG. 10D shows similar cell-surface inhibition results for the mimic antibody characterized in FIG. 10B.

FIGS. 11A-D are summaries showing binding of individual DMS variants of the receptor mimic presented on yeast surface display to (A) a model protein, (B, C) two discovered mimic antibodies, and (D) a known non-mimic antibody. FIGS. 11B and 11C show the two discovered mimic antibodies bind to the mold with similar mutational effect patterns and similar site-specific effects as the model. FIG. 11D shows the known non-mimic antibody binds with a dissimilar pattern in residues 4-30. The Y axis denotes particular substitutions, and the X axis denotes the position along the peptide.

FIGS. 12A-C are line plots showing differential position averaged DMS scores relative to the known non-mimic antibody. FIG. 12A shows the results for the model protein. The model protein contact point residues for the receptor mold were calculated from a published crystal structure of the complex and are highlighted in the grey regions. The model protein DMS scores are increased in the known contact residues relative to the non-mimic antibody. FIG. 12B displays the results for one discovered mimic antibody, showing a similar increase in scores in the known contact regions for the model protein. FIG. 12C displays the results for a second discovered mimic antibody, which likewise showed increase DMS scores in the known contact regions of the model protein.

FIG. 13 depicts scatterplots that show correlations between individual DMS mutation scores between the model protein, the two discovered mimic antibodies and the known non-mimic antibody. Pearson's correlation coefficients for the model protein to the two discovered mimic antibodies was 0.81 and 0.84, respectively. The two sequence-divergent mimic antibodies show a Pearson's correlation coefficient of 0.88. The known non-mimic antibody had correlation coefficients between 0.67-0.68 with the model protein and discovered mimics, showing it has less similar site-specific mutational effects.

### DETAILED DESCRIPTION

The present disclosure is based, in part, on the surprising discovery of a process that facilitates the discovery and identification of multiple classes of mimetic molecules (e.g., mimetic immunoglobulin molecules), which are useful as therapeutics, therapeutic precursors, and/or novel reagents/tool compounds.

However, several challenges have hampered the identification of mimetic molecules, including lack of reliability and lack of ability to scale efficient methods of identification.

Accordingly, the present disclosure provides, in part, a process for identifying mimetic molecules (“mimics”) that is analogous, without wishing to be bound by theory, to the manufacturing process of casting a replica. First, a surface to be mimicked (the model) and a molecule that binds to that surface (the mold) is identified, and then molecules that bind to the mold (candidate mimics) are identified. Finally, in embodiments, detailed characterization of the mold-candidate interactions is performed to identify candidates that imitate the model. In aspects, the present disclosure describes a method of using an anti-antibody as a mold.

Thus, without wishing to be bound by theory, the process of the present disclosure provides distinct advantages in overcoming limitations of the original context for a binding domain by inserting a pharmacophore of an endogenous molecule onto a functional skeleton of a molecule, such as the antigen-binding portion of an immunoglobulin or immunoglobulin-type molecule, such as the paratope of another molecule to copy the endogenous molecule's function with the advantages of the other molecule (e.g., for an antibody, long half-life and straightforward engineering and manufacturing).

In aspects, the present disclosure provides methods of identifying a functional mimic molecule having a target functional profile that comprises one or more functions of a model by (a) providing one or more variant libraries of one or more functional mold molecules of the model, wherein the variant library comprises a plurality of variants of the one or more functional mold molecules, (b) providing one or more candidate functional mimic molecules, (c) evaluating the interaction of the plurality of variant functional mold molecules with (i) the model and (ii) the one or more candidate functional mimic molecules, and comparing the evaluations of i) and ii), and (d) identifying one or more functional mimic molecules having the target functional profile on the basis of the evaluation in c).

In aspects, the present disclosure provides methods of identifying a functional mimic molecule having a target functional profile that comprises one or more functions of a model by (a) providing one or more functional mold molecules of the model, (b) providing one or more variant libraries of one or more candidate functional mimic molecules, the variant libraries comprising a plurality of variants of the one or more candidate functional mimic molecules, (c) evaluating the interaction of the one or more functional mold molecules with: i) the model; and ii) the plurality of variants of the one or more candidate functional mimic molecules, and comparing the evaluations of i) and ii), and (d) identifying one or more functional mimic molecules having the target functional profile on the basis of the evaluation in c).

In aspects, the present disclosure provides methods of identifying a functional mimic molecule having a target functional profile that comprises one or more functions of a model by (a) providing one or more variant libraries of one or more functional mold molecules of the model, the variant libraries comprising a plurality of variants of the one or more functional mold molecules, (b) providing one or more variant libraries of one or more candidate functional mimic molecules, the variant libraries comprising a plurality of variants of the one or more candidate functional mimic molecules, (c) evaluating and comparing the interactions of the plurality of variant functional mold molecules with (i) the model and (ii) the plurality of variants of the one or more candidate functional mimic molecules, and (d) identifying one or more functional mimic molecules having the target functional profile on the basis of the evaluation in c).

The present disclosure further provides, in aspects, methods of identifying functional mimic molecules having target functional profiles that comprise one or more functions of a model by (a) evaluating and comparing the interaction of a plurality of variant functional mold molecules with (i) the model and (ii) one or more candidate functional mimic molecules, or a plurality of variant candidate functional mimic molecules thereof, and (b) identifying one or more functional mimic molecules having the target functional profile on the basis of the evaluation in (a).

### Mimetic (Mimic) Molecules

In embodiments, the present disclosure contemplates that the functional mimic molecule is or comprises an immunoglobulin antibody, an antibody-like molecule, an antibody format, or an antigen-binding fragment thereof. In embodiments, the antibody or antibody-like molecule is selected from a bi-specific antibody, a tri-specific antibody, and an antibody-drug conjugate. In embodiments, the immunoglobulin antibody or antibody-like molecule is monoclonal or polyclonal.

In embodiments, the present disclosure provides mimetic (mimic) immunoglobulin molecules that interact with a binding partner in a way that recapitulates the network of interactions used by another ligand. The mimic immunoglobulin molecule can therefore recapitulate the functional properties of the model ligand by mimicking the structural and/or energetic interactions at a ligand/receptor interface. Thus, in embodiments, the present disclosure provides functional mimic immunoglobulin molecules that exhibit a non-zero similarity to the functional profile of a model. For example, in embodiments, the functional mimic immunoglobulin molecule exhibits a target functional profile having a non-zero similarity to the functional profile of a model, the functional profile comprising one or more of: a structure and a binding profile (e.g., activity and/or affinity, avidity, specificity, and/or selectivity).

In embodiments, the functional mimic immunoglobulin molecule is or comprises an antibody, an antibody format, or an antigen-binding fragment. In embodiments, the antibody is selected from a bi-specific antibody, a tri-specific antibody, and an antibody-drug conjugate. In embodiments, the antibody is monoclonal or polyclonal.

In embodiments, the present disclosure provides that the functional mimic immunoglobulin molecule is an anti-idiotypic antibody, or fragment thereof, that binds the antigen-binding domain of the functional mold immunoglobulin molecule. In embodiments, the anti-idiotypic antibody, or fragment thereof, binds the paratope of the functional mold immunoglobulin molecule. In embodiments, an anti-idiotypic antibody binds to the idiotype of another antibody (e.g., an antibody drug). In embodiments, an idiotype is a specific combination of idiotopes present within an antibody's complement determining regions (CDRs). A single idiotope is a specific region within an antibody's Fv region which binds to the paratope (antigenic epitope binding site) of a different antibody. In embodiments, an idiotope is synonymous with an antigenic determinant of an antibody.

In embodiments, the present disclosure provides that the functional mimic polypeptide is an anti-idiotypic antibody, or fragment thereof, that binds the antigen-binding domain of the functional mold polypeptide. In embodiments, the anti-idiotypic antibody, or fragment thereof, binds the paratope of the functional mold polypeptide. In embodiments, an anti-idiotypic antibody binds to the idiotype of another antibody (e.g., an antibody drug). In embodiments, an idiotype is a specific combination of idiotopes present within an antibody's complement determining regions (CDRs). A single idiotope is a specific region within an antibody's Fv region which binds to the paratope (antigenic epitope binding site) of a different antibody. In embodiments, an idiotope is synonymous with an antigenic determinant of an antibody.

In embodiments, an antibody refers to a broad sense and includes immunoglobulin or antibody molecules including polyclonal antibodies, monoclonal antibodies, including murine, human, humanized and chimeric monoclonal antibodies, and antibody fragments.

In general, antibodies are proteins or polypeptides that exhibit binding specificity to a specific antigen. Intact antibodies are heterotetrametric glycoproteins, composed of two identical light chains and two identical heavy chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa and lambda, based on the amino acid sequences of their constant domains. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4.

In embodiments, an antibody fragment comprises an antigen binding or variable region of an intact antibody. For example, in embodiments, the present disclosure contemplates antibody fragments selected from a single-domain antibody (sdAb), a variable domain of heavy-chain antibodies (VHH), a single-chain antibody (scFv), a shark heavy-chain-only antibody (VNAR), a Fv, a Fab, a Fab′, a F(ab′)₂, and a microprotein (cysteine knot protein, knottin).

In embodiments, an antigen comprises any molecule that has the ability to generate antibodies either directly or indirectly.

In embodiments, a CDR is a complementarity determining region amino acid sequences of an antibody which are the hypervariable regions of immunoglobulin heavy and light chains. See, e.g., Kabat et al., Sequences of Proteins of Immunological Interest, 4th ed., U.S. Department of Health and Human Services, National Institutes of Health (1987). There are three heavy chain and three light chain CDRs or CDR regions in the variable portion of an immunoglobulin. In embodiments, a CDR refers to all three heavy chain CDRs, or all three light chain CDRs or both all heavy and all light chain CDRs, if appropriate.

Each variable region comprises three hypervariable regions also known as complementarity determining regions (CDRs) flanked by four relatively conserved framework regions (FRs). The three CDRs, referred to as CDR1, CDR2, and CDR3, contribute to the antibody binding specificity, as the CDRs provide the majority of contact residues for the binding of the antibody to the antigen or epitope. CDRs of interest can be derived from donor antibody variable heavy and light chain sequences, and include analogs of the naturally occurring CDRs, which analogs also share or retain the same antigen binding specificity and/or neutralizing ability as the donor antibody from which they were derived. In embodiments, the antibody is a chimeric antibody. In embodiments, the antibody is a humanized antibody.

In embodiments, CDRs are based on sequence variability (Wu and Kabat, J. Exp. Med. 132:211-250, 1970). There are six CDRs-three in the variable heavy chain, or VH, and are typically designated H-CDR1, H-CDR2, and H-CDR3, and three CDRs in the variable light chain, or VL, and are typically designated L-CDR1, L-CDR2, and L-CDR3 (Kabat et al., Sequences of Proteins of Immunological Interest, 5th Ed. Public Health Service, National Institutes of Health, Bethesda, Md., 1991). In embodiments, a hypervariable region (“HVR” or “HV”) is a region of an antibody variable domain that is variable in structure as defined by Chothia and Lesk (Chothia and Lesk, Mol. Biol. 196:901-917, 1987). There are six HVRs, three in VH (H1, H2, H3) and three in VL (L1, L2, L3). Chothia and Lesk refer to structurally conserved HVs as canonical structures. Another method of describing the regions that form the antigen-binding site has been proposed by Lefranc (Lefranc et al., Developmental & Comparative Immunology 27:55-77, 2003) based on the comparison of V domains from immunoglobulins and T-cell receptors (Lefranc et al., Developmental & Comparative Immunology 27:55-77, 2003). The antigen-binding site can also be delineated based on Specificity Determining Residue Usage (SDRU), according to Almagro (Almagro, Mol. Recognit. 17:132-43, 2004), where SDRU refers to amino acid residues of an immunoglobulin that are directly involved in antigen contact.

In embodiments, the present disclosure contemplates that the functional mimic molecules can be any suitable class of molecule. In aspects, the present disclosure provides mimetic (mimic) molecules that interact with a binding partner in a way that recapitulates the network of interactions used by another ligand. The mimic molecule can therefore recapitulate the functional properties of the model ligand by mimicking the structural and/or energetic interactions at a ligand/receptor interface.

Thus, in embodiments, the present disclosure provides functional mimic molecules that exhibit a non-zero similarity to the functional profile of a model. For example, in embodiments, the functional mimic molecule exhibits a target functional profile having a non-zero similarity to the functional profile of a model, the functional profile comprising one or more of: a structure, a binding profile (e.g., activity and/or affinity, specificity, and/or selectivity), a signaling profile (activity, specificity, and/or selectivity), an enzymatic profile (activity and/or processivity, specificity, and/or selectivity).

In embodiments, the candidate functional mimic molecule is a small molecule (e.g., a molecular glue). In embodiments, the candidate functional mimic molecule is a small molecule conjugated to another domain (e.g., cereblon, E3-ligase, or immunomodulatory drugs). In embodiments, the molecular glue is conjugated to another domain via click chemistry (e.g., a reaction selected from one or more of conjugate addition, strained ring opening, acylation/sulfonylation, aldehyde capture by a-effect nucleophiles, cycloaddition, and nucleophilic addition to activated alkynes). In embodiments, the candidate functional mimic molecule is a molecular glue. In embodiments, a molecular glue is a type of small molecule stabilizer of protein-protein interactions that encourages two proteins to come together that normally wouldn't interact by changing the surface of their target proteins. In embodiments, molecular glue-induced protein proximity can either be stabilized/activated or destabilized/inactivated, which is dependent on the property of the small molecules and the involved proteins. In embodiments, a molecular glue comprises a molecule selected from one or more of plant hormones, auxin, jasmonate, immunomodulatory imide drugs (IMiDs), e.g., CC-122, CC-220, CC-885, thalidomide, pomalidomide, and lenalidomide. In embodiments, a molecular glue comprises cereblon and/or E3-ligase. In embodiments, the functional mold molecule is a Proteolysis Targeting Chimera (PROTAC), which is a heterobifunctional molecule that forms a ternary complex with the target protein and E3-ligase by making two distinct small molecule13 protein interactions.

In embodiments, the mimic molecule is or comprises a polymer, such as a polypeptide or a nucleic acid, or a combination thereof.

In embodiments, the mimic molecule is or comprises a nucleic acid that is a polynucleotide. In embodiments, the nucleic acid is or comprises DNA, RNA, or a combination thereof. In embodiments, the mimic molecule is a nucleic acid that is a DNA aptamer, a DNA optimer, a RNA aptamer, or a RNA optimer.

In embodiments, DNA or RNA aptamers comprise short, single-stranded DNA or RNA (ssDNA or ssRNA) molecules that can selectively bind to a specific target, including proteins, peptides, carbohydrates, small molecules, toxins, and live cells. Aptamers can assume a variety of shapes due to their tendency to form helices and single-stranded loops and exhibit targeted binding with high selectivity and/or specificity. Aptamers recognize and bind targets as determined by the aptamer's tertiary structure, wherein the aptamer recognizes and binds the target via three-dimensional, shape-dependent interactions, as well as hydrophobic interactions, base-stacking, and intercalation. Aptamers comprising about 15 to about 120 nucleotides can be selected in vitro from a randomized pool of oligonucleotides (about 10¹⁴-about 10¹⁵ molecules). In embodiments, aptamers or aptamer sequences comprise a degenerate sequence, and can further comprise fixed sequences flanking the degenerate sequence. In embodiments, an aptamer further comprises both native and modified DNA and RNA bases, e.g. beta-D-Glucosyl-Hydroxymethyluracil. In embodiments, an optimer is a subset of aptamers.

In embodiments, nucleic acids of the present disclosure (e.g., DNA or RNA aptamers or optimers) comprise non-canonical and/or modified nucleotides. In embodiments, the nucleic acid comprises a chemical modification. In embodiments, the chemical modification is a nucleobase modification, a backbone modification, and/or a sugar modification.

In embodiments, the nucleobase modification replaces A, C, T, G, or U. In embodiments, the nucleobase modification is selected from pseudouridine, N1-methyl-pseudouridine, 5-methylcytidine (m5C), 2′-thiouridine (s2U), N6′-methyladenosine (m6A), and 5′-fluoro-2′-deoxyuridine.

In embodiments, the backbone modification is selected from phosphorothioate, phosphorodithioate, methylphosphonate, and methoxypropylphosphonate.

In embodiments, the sugar modification is selected from 2′-methoxy (2′-OMe), 2′-O-methoxyethyl (2′-O-MOE), 2′-fluoro (2′-F), 2′-arabino-fluoro (2′-Ara-F), 2′-O-benzyl, constrained ethyl (cEt), peptide nucleic acid (PNA), glycol nucleic acid (GNA), unlocked nucleic acid (UNA)and locked nucleic acid (LNA).

In embodiments, nucleic acid molecules of the present disclosure may include one or more modified nucleotides. Exemplary modified nucleotides are described in U.S. Pat. No. 8,278,036, which is hereby incorporated by reference in its entirety. In embodiments, the modified nucleotides may be selected from one or more of pseudouridine, N1-methylpseudouridine, 5-methylcytidine, or N6-methyladenosine, 5-hydroxycytidine, 5-hydroxy methylcytidine, 5-carboxycytidine, 5-formylcytidine, 5-hydroxyuridine, 5-hydroxymethyluridine, 5-carboxyuridine, 5-formyluridine, pseudouridine, 2-thiouridine, 4-thiouridine, 5-azauridine, 5-aminouridine, 5-methyluridine, 2-thiopseudouridine, 4-thiopseudouridine, 5-hydroxypseudouridine, 5-methylpseudouridine, 5-aminopseudouridine, pseudoisocytidine, N4-methylcytidine, 2-thiocytidine, 5-azacytidine, 5-aminocytidine, N4-methylpseudoisocytidine, 2-thiopseudoisocytidine, 5-hydroxypseudoisocytidine, 5-aminopseudoisocytidine, 5-methylpseudoisocytidine, 7-deazaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

In embodiments, nucleic acids are chemically modified. In embodiments, the chemical modification is selected from pseudouridine, N1-methyl-pseudouridine, 5-methylcytidine (m5C), 2′-thiouridine (s2U), N6′-methyladenosine (m6A), and 5′-fluoro-2′-deoxyuridine, phosphorothioate, phosphorodithioate, methylphosphonate, methoxypropylphosphonate, 2′-methoxy (2′-OMe), 2′-O-methoxyethyl (2′-O-MOE), 2′-fluoro (2′-F), 2′-arabino-fluoro (2′-Ara-F), 2′-O-benzyl, constrained ethyl (cEt), peptide nucleic acid (PNA), glycol nucleic acid (GNA), unlocked nucleic acid (UNA)and locked nucleic acid (LNA).

In embodiments, DNA or RNA aptamers or optimers of the present disclosure comprise non-canonical base pairing, which occurs when nucleobases hydrogen bond, or base pair, to one another in schemes other than the standard Watson-Crick base pairs (e.g., adenine (A)—thymine (T) in DNA; adenine (A)—uracil (U) in RNA; and guanine (G)—cytosine (C) in both DNA and RNA). For example, non-canonical DNA structures can include, but are not limited to, A-DNA, B-DNA, Z-DNA, hairpin/cruciform, triplex, G-quadruplex, and/or i-motif.

In embodiments, the contemplated mimic molecule is or comprises a peptide nucleic acid that includes an altered deoxyribose phosphate backbone. Peptide nucleic acids (PNAs) are synthetic mimics of DNA in which the deoxyribose phosphate backbone is replaced by repetitive units of N-(2-aminoethyl) glycine to which the purine and pyrimidine bases are attached via a methyl carbonyl linker. Peptide nucleic acids can hybridize with complementary DNAs or RNAs with remarkably high affinity and specificity, due to their uncharged and flexible polyamide backbone. Peptide nucleic acids hybridize to complementary DNA or RNA in a sequence-dependent manner, according to the Watson-Crick hydrogen bonding scheme. In contrast to DNA, a peptide nucleic acid can bind in either parallel or antiparallel manner. Peptide nucleic acids are able to adopt both A-type and B-type structures when associating with RNA and DNA, respectively, whereas PNA-PNA duplexes form an unusual helix conformation, called P-type and are characterized by a large pitch of 18 base pairs. Peptide nucleic acids, and their unique properties as DNA mimics, can be used in drug design (e.g., gene therapy drug design), as well as in prognostics, diagnostics, and disease monitoring.

In embodiments, the nucleic acid is or comprises a ribozyme and/or riboswitch. In embodiments, a riboswitch is a regulatory segment of a messenger RNA molecule that binds a small molecule.

In embodiments, the present disclosure contemplates that the mimic molecule is or comprises a polypeptide—that is, the mimic is a functional mimic polypeptide. In embodiments, the functional mimic polypeptide comprises one or more non-canonical or unnatural amino acids. For example, a non-canonical or unnatural amino acid is a non-proteinogenic amino acid that is either found naturally in organisms or is synthetically made in a laboratory. A non-canonical or unnatural amino acid is an amino acid that is not located in the genetic code of naturally occurring organisms. In embodiments, a non-canonical or unnatural amino acid is selected from selenocysteine, pyrrolysine, N-formylmethionine-alanine, GABA and 8-Aminolevulinic acid, 4-aminobenzoic acid (PABA), D-isomers of the common amino acids, 2,4-diaminobutyric acid, 60 -amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general.

In embodiments, the functional mimic polypeptide comprises one or more L-amino acids (L-isomer of a canonical or non-canonical amino acid) and/or D-amino acids (D-isomer of a canonical or non-canonical amino acid). In embodiments, the functional mimic polypeptide comprises non-canonical or unnatural amino acid comprising a L-amino acid or a D-amino acid.

In embodiments, the present disclosure contemplates a functional mimic polypeptide that is a binding agent. In embodiments, the binding agent is or comprises an immunoglobulin antibody, an antibody-like molecule, or an antigen-binding fragment thereof.

In embodiments, the functional mimic polypeptide is a binding agent that comprises a non-immunoglobulin-based binding domain. In embodiments, the non-immunoglobulin-based binding domain is selected from an affimer, an affilin, an affitin, an affibody, an alphabody, an anticalin, an avimer, a DARPin, a fynomer, a gastrobody, a Kunitz domain, a monobody, a nanoCLAMP, a repebody, a pronectin, a centyrin, and an obody.

In embodiments, the functional mimic polypeptide comprises a non-immunoglobulin-based binding domain that is or comprises an enzyme, or a fragment thereof.

In embodiments, the functional mimic polypeptide comprises a non-immunoglobulin-based binding domain that is or comprises a signaling protein, or a fragment thereof. In embodiments, the signaling protein is selected from a cytokine, growth factor, or hormone.

In embodiments, the functional mimic polypeptide comprises a non-immunoglobulin-based binding domain that is or comprises a structural protein such as a receptor, or a fragment thereof.

In embodiments, the functional mimic polypeptide comprises a non-immunoglobulin-based binding domain that is or comprises a ligand.

In embodiments, the functional mimic polypeptide comprises a non-immunoglobulin-based binding domain that is or comprises an extracellular domain.

In embodiments, the functional mimic polypeptide comprises a non-immunoglobulin-based binding domain that is or comprises an extracellular domain.

In embodiments, the functional mimic polypeptide is or comprises a bacterial nucleic acid binding protein, such as a bacteriophage MS2 protein, which binds to a small RNA hairpin in its genomic RNA, or a bacteriophage lambda N protein, which binds to a boxB hairpin loop.

In embodiments, the binding agent is bifunctional. For example, in embodiments, the binding agent comprises a fusion protein or a non-fusion protein conjugate. In embodiments where the binding agent is a conjugate, the binding agent is conjugated to a carbohydrate (e.g., polysaccharide), a lipid, or a small molecule. In embodiments, the binding agent is glycosylated, lapidated, or otherwise conjugated.

The present disclosure provides methods for identifying one or more functional mimic molecules (e.g., functional mimic polypeptides). For example, functional mimic molecules (e.g., functional mimic polypeptides) can be identified by biopanning, anti-drug antibody (ADA) repertoire mining, reported pertinent interactions with other molecules and/or complexes; phage display; immunization (e.g., of a mammal, such as a mouse, rat, rabbit, non-human primate (NHP), or human) with the model; obtaining model-binding B cells from a subject previously exposed, or suspected of being exposed, to the model; competitive binding assays; depletion studies; and/or structure-based modeling (e.g., de novo in silico molecule design). In embodiments, immunization of a mammal (e.g., a mouse, rat, rabbit, NHP, or human) with the model further comprises isolating model-binding B-cells and performing B cell receptor (BCR) sequencing. BCR sequencing employ high-throughput single-cell sequencing to obtain the VH and VL sequences for antibodies from individual human, rat, and mouse B cells.

### Model Molecule or Complex

The present disclosure provides, in embodiments, for a model molecule or complex that comprises a surface to be mimicked. In embodiments, a model is a reference molecule or complex to be functionally mimicked by a functional mimic molecule.

In embodiments, a model is non-limiting and can be any molecule or complex. In embodiments, a model may be a polypeptide or a non-polypeptide (e.g., a small molecule or nucleic acid). For example, a model can be a protein, a peptide, a lipid, a carbohydrate, a nucleic acid, or an inorganic molecule.

In embodiments, the model comprises an antigen, which can be selected from a viral protein, a bacterial protein, a protozoan protein, an Archean protein, a fungal protein, a mammalian protein, or an inorganic protein, a small molecule, a carbohydrate, a nucleic acid, a lipid, or a plastic.

In embodiments, the model is a therapeutic molecule. For example, the model can be a therapeutic antibody or antigen-binding fragment thereof. In embodiments, the antigen-binding fragment of the antibody is the paratope of the antibody.

### Mold Molecule

The present disclosure contemplates mold molecules that bind to the surface to be mimicked (the “model”), and which are bound by candidate mimic molecules.

In embodiments, a functional mold molecule is a molecule that exhibits some non-zero functional complementarity to a model and/or a functional mimic polypeptide. In embodiments, functional complementarity refers to binding. By way of illustration, in embodiments, a functional mold polypeptide is or comprises an antibody that binds the model (e.g., antigen), and the functional mimic polypeptide is or comprises an anti-idiotype antibody (or fragment thereof) that binds the antigen-binding domain of the functional mold polypeptide, specifically the paratope.

In embodiments, the present disclosure provides that the functional mold molecule is a functional mold polypeptide, such as an antibody, and the functional complementarity comprises binding to the model (e.g., an antigen). In embodiments, the functional mimic polypeptide is an anti-idiotypic antibody, or fragment thereof, that binds the antigen-binding domain of the functional mold polypeptide, specifically the paratope.

In embodiments, the functional mold molecule is a small molecule (e.g., a molecular glue). In embodiments, the functional mold molecule is a small molecule conjugated to another domain (e.g., cereblon, E3-ligase, or immunomodulatory drugs). In embodiments, the molecular glue is conjugated to another domain via click chemistry (e.g., a reaction selected from one or more of conjugate addition, strained ring opening, acylation/sulfonylation, aldehyde capture by a-effect nucleophiles, cycloaddition, and nucleophilic addition to activated alkynes). In embodiments, the functional mold molecule is a molecular glue. In embodiments, a molecular glue is a type of small molecule stabilizer of protein-protein interactions that encourages two proteins to come together that normally wouldn't interact by changing the surface of their target proteins. In embodiments, molecular glue-induced protein proximity can either be stabilized/activated or destabilized/inactivated, which is dependent on the property of the small molecules and the involved proteins. In embodiments, a molecular glue comprises a molecule selected from one or more of plant hormones, auxin, jasmonate, immunomodulatory imide drugs (IMiDs), e.g., CC-122, CC-220, CC-885, thalidomide, pomalidomide, and lenalidomide. In embodiments, a molecular glue comprises cereblon and/or E3-ligase. In embodiments, the functional mold molecule is a Proteolysis Targeting Chimera (PROTAC), which is a heterobifunctional molecule that forms a ternary complex with the target protein and E3-ligase by making two distinct small molecule-protein interactions.

In embodiments, a functional mold molecule is non-limiting and can be any class of molecule, such as a polymer. In embodiments, the polymer is a polypeptide, a nucleic acid, or a combination thereof.

In embodiments, the functional mold molecule is or comprises a nucleic acid that is a DNA aptamer, a DNA optimer, a RNA aptamer, or a RNA optimer. In embodiments, DNA or RNA aptamers comprise short, single-stranded DNA or RNA (ssDNA or ssRNA) molecules that can selectively bind to a specific target, including proteins, peptides, carbohydrates, small molecules, toxins, and live cells. Aptamers can assume a variety of shapes due to their tendency to form helices and single-stranded loops and exhibit targeted binding with high selectivity and/or specificity. Aptamers recognize and bind targets as determined by the aptamer's tertiary structure, wherein the aptamer recognizes and binds the target via three-dimensional, shape-dependent interactions, as well as hydrophobic interactions, base-stacking, and intercalation. Aptamers comprising 15 to 120 nucleotides can be selected in vitro from a randomized pool of oligonucleotides (10¹⁴-10¹⁵ molecules). In embodiments, aptamers or aptamer sequences comprise a degenerate sequence, and can further comprise fixed sequences flanking the degenerate sequence. In embodiments, an aptamer further comprises both native and modified DNA and RNA bases, e.g. beta-D-Glucosyl-Hydroxymethyluracil. In embodiments, an optimer comprises a subset of aptamers.

In embodiments, nucleic acids of the present disclosure (e.g., DNA or RNA aptamers or optimers) comprise non-canonical and/or modified nucleotides. In embodiments, the nucleic acid comprises a chemical modification. In embodiments, the chemical modification is a nucleobase modification, a backbone modification, and/or a sugar modification.

In embodiments, the nucleobase modification replaces A, C, T, G, or U. In embodiments, the nucleobase modification is selected from pseudouridine, N1-methyl-pseudouridine, 5-methylcytidine (m5C), 2′-thiouridine (s2U), N6′-methyladenosine (m6A), and 5′-fluoro-2′-deoxyuridine.

In embodiments, the backbone modification is selected from phosphorothioate, phosphorodithioate, methylphosphonate, and methoxypropylphosphonate.

In embodiments, the sugar modification is selected from 2′-methoxy (2′-OMe), 2′-O-methoxyethyl (2′-O-MOE), 2′-fluoro (2′-F), 2′-arabino-fluoro (2′-Ara-F), 2′-O-benzyl, constrained ethyl (cEt), peptide nucleic acid (PNA), glycol nucleic acid (GNA), unlocked nucleic acid (UNA)and locked nucleic acid (LNA).

In embodiments, nucleic acid molecules of the present disclosure may include one or more modified nucleotides. Exemplary modified nucleotides are described in U.S. Pat. No. 8,278,036, which is hereby incorporated by reference in its entirety. In embodiments, the modified nucleotides may be selected from one or more of pseudouridine, N1-methylpseudouridine, 5-methylcytidine, or N6-methyladenosine, 5-hydroxycytidine, 5-hydroxymethylcytidine, 5-carboxycytidine, 5-formylcytidine, 5-hydroxyuridine, 5-hydroxymethyluridine, 5-carboxyuridine, 5-formyluridine, pseudouridine, 2-thiouridine, 4-thiouridine, 5-azauridine, 5-aminouridine, 5-methyluridine, 2-thiopseudouridine, 4-thiopseudouridine, 5-hydroxypseudouridine, 5-methylpseudouridine, 5-aminopseudouridine, pseudoisocytidine, N4-methylcytidine, 2-thiocytidine, 5-azacytidine, 5-aminocytidine, N4-methylpseudoisocytidine, 2-thiopseudoisocytidine, 5-hydroxypseudoisocytidine, 5-aminopseudoisocytidine, 5-methylpseudoisocytidine, 7-deazaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

In embodiments, nucleic acids are chemically modified. In embodiments, the chemical modification is selected from pseudouridine, N1-methyl-pseudouridine, 5-methylcytidine (m5C), 2′-thiouridine (s2U), N6′-methyladenosine (m6A), and 5′-fluoro-2′-deoxyuridine, phosphorothioate, phosphorodithioate, methylphosphonate, methoxypropylphosphonate, 2′-methoxy (2′-OMe), 2′-O-methoxyethyl (2′-O-MOE), 2′-fluoro (2′-F), 2′-arabino-fluoro (2′-Ara-F), 2′-O-benzyl, constrained ethyl (cEt), peptide nucleic acid (PNA), glycol nucleic acid (GNA), unlocked nucleic acid (UNA)and locked nucleic acid (LNA).

The DNA or RNA aptamers or optimers can include non-canonical and/or modified nucleotides. In embodiments, DNA or RNA aptamers or optimers of the present disclosure comprise non-canonical base pairing, which occurs when nucleobases hydrogen bond, or base pair, to one another in schemes other than the standard Watson-Crick base pairs (e.g., adenine (A)—thymine (T) in DNA; adenine (A)—uracil (U) in RNA; and guanine (G)—cytosine (C) in both DNA and RNA). For example, non-canonical DNA structures can include, but are not limited to, A-DNA, B-DNA, Z-DNA, hairpin/cruciform, triplex, G-quadruplex, and/or i-motif.

In embodiments, the contemplated functional mold molecule is or comprises a peptide nucleic acid that includes an altered deoxyribose phosphate backbone. Peptide nucleic acids (PNAs) are synthetic mimics of DNA in which the deoxyribose phosphate backbone is replaced by repetitive units of N-(2-aminoethyl) glycine to which the purine and pyrimidine bases are attached via a methyl carbonyl linker. Peptide nucleic acids can hybridize with complementary DNAs or RNAs with remarkably high affinity and specificity, due to their uncharged and flexible polyamide backbone. Peptide nucleic acids hybridize to complementary DNA or RNA in a sequence-dependent manner, according to the Watson-Crick hydrogen bonding scheme. In contrast to DNA, a peptide nucleic acid can bind in either parallel or antiparallel manner. Peptide nucleic acids are able to adopt both A-type and B-type structures when associating with RNA and DNA, respectively, whereas PNA-PNA duplexes form an unusual helix conformation, called P-type and are characterized by a large pitch of 18 base pairs. Peptide nucleic acids, and their unique properties as DNA mimics, can be used in drug design (e.g., gene therapy drug design), as well as in prognostics, diagnostics, and disease monitoring.

In embodiments, the present disclosure contemplates that the functional mold molecule is or comprises a polypeptide—that is, the mold molecule is a functional mold polypeptide. In embodiments, the functional mold polypeptide comprises one or more non-canonical or unnatural amino acids. For example, a non-canonical or unnatural amino acid is a non-proteinogenic amino acid that is either found naturally in organisms or is synthetically made in a laboratory. A non-canonical or unnatural amino acid is an amino acid that is not located in the genetic code of naturally occurring organisms. In embodiments, a non-canonical or unnatural amino acid is selected from selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general. In embodiments, the functional mold polypeptide comprises one or more L-amino acids (L-isomer of a canonical or non-canonical amino acid) and/or D-amino acids (D-isomer of a canonical or non-canonical amino acid). In embodiments, the functional mold polypeptide comprises non-canonical or unnatural amino acid comprising a L-amino acid or a D-amino acid.

In embodiments, the present disclosure contemplates a functional mold polypeptide that is a binding agent. In embodiments, the binding agent comprises an antibody or antibody format, or a fragment (e.g., an antigen-binding fragment) thereof. In embodiments, the binding agent comprises a non-immunoglobulin-based binding domain.

In embodiments, an antibody refers to a broad sense and includes immunoglobulin or antibody molecules including polyclonal antibodies, monoclonal antibodies, including murine, human, humanized and chimeric monoclonal antibodies, and antibody fragments. In general, antibodies are proteins or polypeptides that exhibit binding specificity to a specific antigen. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4.

In embodiments, an antibody fragment comprises an antigen binding or variable region of an intact antibody. For example, in embodiments, the present disclosure contemplates antibody fragments selected from a single-domain antibody (sdAb), a variable domain of heavy-chain antibodies (VHH), a single-chain antibody (scFv), a shark heavy-chain-only antibody (VNAR), a Fv, a Fab, a Fab′, a F(ab′)₂, and a microprotein (cysteine knot protein, knottin).

In embodiments, an antigen comprises any molecule that has the ability to generate antibodies either directly or indirectly.

In embodiments, the functional mold polypeptide is a binding agent that comprises a non-immunoglobulin-based binding domain. In embodiments, the non-immunoglobulin-based binding domain is selected from an affimer, an affilin, an affitin, an affibody, an alphabody, an anticalin, an avimer, a DARPin, a fynomer, a gastrobody, a Kunitz domain, a monobody, a nanoCLAMP, a repebody, a pronectin, a centyrin, and an obody.

In embodiments, the functional mold polypeptide comprises a non-immunoglobulin-based binding domain that is or comprises an enzyme, or a fragment thereof.

In embodiments, the functional mold polypeptide comprises a non-immunoglobulin-based binding domain that is or comprises a signaling protein, or a fragment thereof. In embodiments, the signaling protein is selected from a cytokine, growth factor, or hormone.

In embodiments, the functional mold polypeptide comprises a non-immunoglobulin-based binding domain that is or comprises a structural protein such as a receptor, or a fragment thereof.

In embodiments, the functional mold polypeptide comprises a non-immunoglobulin-based binding domain that is or comprises a ligand.

In embodiments, the functional mold polypeptide comprises a non-immunoglobulin-based binding domain that is or comprises an extracellular domain.

In embodiments, the functional mold polypeptide comprises a non-immunoglobulin-based binding domain that is or comprises an extracellular domain.

In embodiments, the functional mold polypeptide is or comprises a bacterial nucleic acid binding protein, such as a bacteriophage MS2 protein, which binds to a small RNA hairpin in its genomic RNA, or a bacteriophage lambda N protein, which binds to a boxB hairpin loop.

In embodiments, the binding agent is bifunctional. For example, in embodiments, the binding agent comprises a fusion protein or a non-fusion protein conjugate. In embodiments where the binding agent is a conjugate, the binding agent is conjugated to a carbohydrate (e.g., polysaccharide), a lipid, or a small molecule. In embodiments, the binding agent is glycosylated, lapidated, or otherwise conjugated.

The present disclosure provides methods for identifying one or more functional mold molecules (e.g., functional mold polypeptides). For example, functional mold molecules (e.g., functional mold polypeptides) can be identified by reported pertinent interactions with other molecules and/or complexes; phage display; immunization (e.g., of a mammal, such as a mouse, rat, rabbit, non-human primate (NHP), or human) with the model; obtaining model-binding B cells from a subject previously exposed, or suspected of being exposed, to the model; competitive binding assays; depletion studies; and/or structure-based modeling (e.g., de novo in silico molecule design). In embodiments, immunization of a mammal (e.g., a mouse, rat, rabbit, NHP, or human) with the model further comprises isolating model-binding B-cells and performing B cell receptor (BCR) sequencing. BCR sequencing employ high-throughput single-cell sequencing to obtain the VH and VL sequences for antibodies from individual human, rat, and mouse B cells.

In embodiments, the functional mold molecule is a functional mold polypeptide comprising functional complementarity. In embodiments, the functional mold polypeptide binds the model or the candidate mimic molecule. In embodiments, the functional mold molecule's binding to the model or the candidate mimic molecule is measured via binding affinity interaction assays. In embodiments, the binding affinity interactions are measured by one or more of a chemical assay, an optical assay, and a radioactive assay. In embodiments, the chemical assay is selected from one or more of gel electrophoresis, enzyme-linked immunosorbent assay (ELISA), and immunoblotting (e.g., a western blot). In embodiments, the optical assay is selected from one or more of a fluorescence intensity assay, a fluorescent anisotropy assay, and a fluorescence resonance energy transfer (FRET) assay. In embodiments, the radioactive binding assay is a G protein-coupled receptor (GPCR)-radioligand binding assay.

### Variant Libraries

The present disclosure contemplates generating and evaluating variant molecules (e.g., mimic molecules and/or variant mold molecules). Accordingly, in embodiments, variant libraries of molecules are generated and/or provided. In embodiments, variant libraries of mimic molecules; mold molecules; and/or both mimic molecules and mold molecules are generated and/or provided.

In embodiments, a variant library is or comprises a collection of variants of some reference molecules, such as polypeptides, across the entire molecule (e.g., polypeptide), or in embodiments, a subset (e.g., a contiguous domain or a non-contiguous set) of positions (e.g., residues) of the molecule.

In embodiments, the variant library comprises a collection of variants that is systematically engineered. In embodiments, the systematic protein engineering comprises a method selected from combinatorial alanine-scanning strategy, binomial mutagenesis, and shotgun scanning. In embodiments, the systematic protein engineering comprises site-directed mutagenesis.

An alanine-scanning strategy is a form of site-directed mutagenesis that allows for systematic mapping of functional binding epitopes. Because substitution with alanine removes all side chain atoms past the β-carbon, the effects of individual alanine mutations can be used to infer the roles of individual side chains. Alanine-scanning mutagenesis provides a detailed map of a protein-binding interface.

Binomial mutagenesis is a combinatorial mutagenesis technique that allows the effects of a given mutation to be studied in the context of many other mutations, thereby allowing for an analysis of additivity and potential residue-residue interactions. For example, when a residue such as alanine is substituted for the wild-type amino acid simultaneously at a set of N positions with a given probability, the randomization results in a set of mutant proteins with a binomial distribution of substitutions. The mutants are then classified into active and inactive classes, and subsets of both classes are then sequenced. The importance of the wild-type residue at a given position can be inferred from the frequency with which the mutation to alanine occurs in the set of active mutants. In principle, if the change is completely neutral, alanine and wild type should each occur in roughly half of the active mutants. A deleterious mutation will be recovered less frequently and vice versa. The frequency with which mutations occur at two positions simultaneously can also be compared with the frequency expected from the product of the single-position frequencies.

Shotgun scanning is a general combinatorial method for rapidly mapping functional epitopes of proteins by combining the concepts of alanine-scanning mutagenesis and binomial mutagenesis with phage display technology. Specifically, shotgun scanning analyzes many side chains simultaneously and circumvents the need for protein purification and biophysical analysis by instead deriving the energetic contributions of individual side chains from statistical analysis of DNA sequences.

In embodiments, the variant library comprises a collection of variants that is random or semi-systematically engineered. In embodiments, the method employs fully random mutation and/or random mutation focused on a specific set of positions (e.g., residues) of the molecule.

In embodiments, a variant is a molecule having one or more of: a substitution, an insertion, or a deletion at one or more positions, relative to a reference molecule, such as a polymer (e.g., a polypeptide). In embodiments, variants are those that have conservative amino acid substitutions made at one or more predicted non-essential amino acid residues. For example, a conservative amino acid substitution is one in which the amino acid residue is replaced with an amino acid residue having a similar side chain.

In embodiments, the amino acid mutations are amino acid substitutions, and may include conservative and/or non-conservative substitutions.

Conservative substitutions may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

In embodiments conservative substitutions are exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt a-helices.

In embodiments, non-conservative substitutions are exchanges of an amino acid by another amino acid listed in a different group of the six standard amino acid groups (1) to (6) shown above.

In embodiments, the variant library comprises a collection of variant polypeptide molecules, and the variant library is one or more of: an alanine (or other fixed amino acid substitution) library, a subset of possible substitutions library, or a full-substitution (including deletions and insertions) library.

In embodiments, the variant library comprises variation across the entirety of a reference molecule (e.g., a polypeptide). In embodiments, the variant library comprises variation across a subset of residues of a reference molecule (e.g., a polypeptide).

In embodiments, the variant library covers at least about 5%, about 10%, about 15%, about 20%, about 25%, about 30%, about 35%, about 40%, about 45%, about 50%, about 55%, about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or more of the contiguous or non-contiguous average length of a functional molecule (e.g., a functional mold molecule or functional mimic molecule), wherein the average length of a functional molecule is at least about 10, about 20, about 30, about 40, about 50, about 60, about 70, about 80, about 90, about 100, about 110, about 120, about 130, about 140, or about 150 monomers (e.g., amino acids or nucleotides), and/or comprises at least about 100, about 200, about 400, about 500, about 1000, about 2500, about 5000, about 10000, about 25000, about 30000, about 40000, about 50000, or about 60000 distinct sequences. In embodiments, the variant library, relative to a reference polymer (e.g., polypeptide), comprises at least about 80%, about 85%, about 90%, about 95%, or about 100% single position substitutions to a specific residue (e.g., alanine), at least about 80%, about 85%, about 90%, about 95%, or about 100% single position substitutions to any residue (e.g., any non-identity residue of the 20 canonical amino acids, including deletions or insertions of at least about 1, about 2, about 3, about 4, about 5, about 6, about 7, about 8, about 9, about 10, or more residues), or combinations thereof, e.g., n% of all double substitutions, m% of all triple substitutions, or approximately following a negative binomial distribution with shape parameters approximately described by probability ~1−0.4*n_sucesses^{-3/4}. In embodiments, the variant library comprises one or more immunoglobulin variable domains (variable heavy, variable light, or variable heavy and variable light), or fragments thereof (e.g., hCDR 1, hCDR 2, hCDR 3, including combinations thereof, including 1, 2, or all 3, e.g., hCDRs1-3; or ICDR 1, 1CDR 2, 1CDR 3, including combinations thereof, including 1, 2, or all 3, e.g., ICDRs1-3). In embodiments, the fragment comprises 3 or 6 CDRs.

In embodiments, the variant library comprises all possible single mutations according to any design and a distribution of about 2, about 3, about 4, or more multiple mutants. In embodiments, the variant library is a deep mutational scan (DMS) library.

Deep mutational scanning is a method for multiplex measurement of functional consequences of protein variants. Specifically, DMS assays can investigate protein-ligand binding affinity by measuring the functional consequences of hundreds of thousands of variants of a protein simultaneously. In a deep mutational scan, a library of protein variants is first introduced into a model system. Model systems that have been used in deep mutational scanning include phage, bacteria, yeast, and cultured mammalian cells. A selection is applied for protein function or another molecular property of interest, altering the frequency of each variant according to its functional capacity. Selections can be growth-based or implement physical separation of variants into bins, as in phage display or flow sorting of cells. Next, the frequency of each variant in each time point or bin is determined by using deep sequencing to count the number of times each variant appears. The variable region is either directly sequenced using a single-end or paired-end strategy, or a short barcode that uniquely identifies each variant in the population is sequenced instead. Barcoding enables accurate assessment of variable regions longer than a single sequencing read. Analysis of the change in each variant's frequency throughout the selection yields a score that estimates the variant's effect. Essentially, in DMS experiments, one begins with a library of variants of a specific protein. Proteins that have high levels of a particular activity of interest are then enriched via one or more rounds of selection. The set of enriched sequences is then compared to the initial library, and protein sequences (or mutations within these sequences) are scored according to how much this enrichment procedure increases their prevalence.

In embodiments, the present disclosure provides a variant library of functional mold molecules (e.g., polypeptides) and/or functional mimic molecules (e.g., candidate functional mimic polypeptides).

In embodiments, providing one or more variant libraries of one or more functional mold molecules or functional mimic molecules further comprises generation of the one or more functional mold molecules or functional mimic molecules by a method selected from interactions reported in the scientific literature, phage display, ribosome display, mammalian cell display, immunization (e.g., of a mammal, such as a mouse, rat, rabbit, non-human primate, or human) with the model (optionally by isolating model-binding B-cells and performing BCR sequencing), obtaining model-binding B cells from a subject previously exposed (or suspected of being exposed) to the model, competitive-binding assays, depletion studies, and structure-based modeling (including de novo in silico molecule design).

In embodiments, the variant library is provided as a collection of one or more soluble proteins and/or cell surface displayed constructs and/or phage display. In embodiments, the soluble proteins of the variant library are full Ig, or fragments thereof.

In embodiments, the cell surface displayed constructs comprises a yeast cell, a mammalian cell, an immortalized cell, or an insect cell.

In embodiments, the yeast cell is selected from Saccharomyces cerevisiae, Schizosaccharomyces pombe, Pichia pastoris, Hansenula polymorpha, Yarrowia lipolytica, and Kluyveromyces lactis.

In embodiments, the mammalian cell is a human cell. In embodiments, the mammalian cell is selected from a Chinese hamster ovary (CHO) cell, a baby hamster kidney (BHK) cell, a human embryonic kidney (HEK293T) cell, a Vero cell, or a Spodoptera frugiperda 9 (Sf9) cell. In embodiments, the CHO cell is a CHO-K1, a CHO-DHB11, a CHO-DXB1, a CHO-S, or a CHO-DG44 cell. In embodiments, the CHO cell comprises or is selected from CHO-K1 (ATCC CCL-61) cells, SURE CHO-M cells (derivative of CHO-K1), or baby hamster kidney cells (BHK, ATCC CCL-10). In embodiments, the Vero cell comprises or is selected from a Vero cell, a Vero 76 cell, and a Vero E6 cell. In embodiments, the cell is or comprises a human cervical carcinoma cell (HELA, ATCC CCL-2), 293 (ATCC CRL-1573), 3T3 (ATCC CCL-163), or a monkey kidney CV1 line (ATCC CCL-70), which can be transformed with SV40 (COS-7, ATCC CRL-1587).

In embodiments, elements of the variant library are expressed individually or as a pool.

### Target Functional Profile

The present disclosure contemplates a target functional profile expressed by a molecule or complex of the disclosure. In embodiments, a target functional profile is or comprises a description of the functions of a molecule or complex (e.g., a functional mimic molecule), relative to a model molecule or complex. For example, in embodiments, illustrative functions include, but are not limited to, affinity for a receptor or ligand; molecular structure or surface features, such as arrangements of charge, polarity, or hydrophobicity; and biological activity, such as modulation of cellular signaling, communication, or pathogen interactions. In embodiments, the target functional profile comprises functions selected from a binding profile (e.g., activity and/or affinity, specificity, and/or selectivity); a signaling profile (activity, specificity, and/or selectivity); and an enzymatic profile (activity and/or processivity, specificity, and/or selectivity).

In embodiments, the target functional profile comprises one or more functions of the mimic molecule or complex, relative to the model. In embodiments, the function is selected from one or more of the affinity, avidity, activity, specificity, and/or selectivity for a receptor or ligand; agonism, antagonism, and/or inhibition; the molecular structure or surface features; and biological activity. In embodiments, the molecular structure or surface features comprises arrangements of charge, polarity, and/or hydrophobicity. In embodiments, the biological activity comprises modulation of cellular signaling, communication, and/or pathogen interactions. In embodiments, the biological activity comprises transcription, translation, and/or post-translational modification.

In embodiments, the functional mimic molecule exhibits a target functional profile having a non-zero similarity to the functional profile of a model, the functional profile comprising one or more of: a structure, a binding profile (e.g., activity and/or affinity, avidity, specificity, and/or selectivity), a signaling profile (activity, specificity, and/or selectivity), an enzymatic profile (activity and/or processivity, specificity, and/or selectivity).

### Characterization of Interactions

The present disclosure provides for a detailed characterization of one or more mold-candidate mimic interactions that was performed in order to identify candidate mimics that imitate the model.

In embodiments, binding affinity interactions of one or more mold-candidate mimic interactions or mold-model interactions can be measured by chemical assays, optical assays, radioactive assays.

In embodiments, chemical binding assays can measure interactions between two molecules (e.g., protein binding another protein, a small molecule, or a nucleic acid). In embodiments, a chemical assay is selected from one or more of gel electrophoresis, enzyme-linked immunosorbent assay (ELISA), and immunoblotting. In embodiments, the immunoblot is a western blot.

In embodiments, optical binding assays can measure interactions between two molecules (e.g., protein binding another protein, a small molecule, or a nucleic acid). In embodiments, the optical assay is selected from one or more of a fluorescence intensity assay, a fluorescent anisotropy assay, and a fluorescence resonance energy transfer (FRET) assay.

In embodiments, the radioactive binding assay is a radioimmunoassay, such as a radioallergosorbent test. In embodiments, a radioimmunoassay is a heterogeneous assay that employs a radiolabeled drug, such as isotopes of iodine (e.g., 1251, 1311, or tritium (3H)) as labels.

In embodiments, the binding affinity interactions are measured individually by flow cytometry, biolayer interferometry, surface plasmon resonance, or enzyme-linked immunosorbent assay.

In embodiments, the method of the present disclosure further comprises an interaction evaluation that provides a first vector comprising a model fingerprint and a second vector comprising a functional mimic molecule fingerprint, wherein the evaluation constitutes calculating a metric of similarity between the vectors. In embodiments, a fingerprint is a set of calculated characteristics for measuring an interaction between vectors. In embodiments, a fingerprint is a set of calculated characteristics for measuring an interaction between vectors using one or more of the methods described herein.

In embodiments, the interaction evaluation is of binding affinity of the variant functional mold molecules and/or of the variant functional mimic molecules to i) the target and ii) the one or more functional mimic molecules and/or functional mold molecules, respectively.

In embodiments, the interaction evaluation comprises evaluating binding affinity interactions, wherein the binding affinity interactions are measured individually. In such embodiments, the individual measurement comprises one or more of flow cytometry, biolayer interferometry, surface plasmon resonance, and/or enzyme-linked immunosorbent assay.

In embodiments, the interaction evaluation comprises evaluating binding affinity interactions, wherein the binding affinity interactions are measured as a pool, where candidate functional mimic molecules are conjugated to a fluorophore or magnetic bead and used to stain cells expressing variants of functional mold molecules as surface display constructs. In embodiments, bound and unbound populations of functional mimic molecules are separated by fluorescence-activated cell sorting (FACS) or magnetic-activated cell sorting. In embodiments, antibody DNA sequences are amplified from the unsorted, unsorted, and/or bound cell populations by polymerase chain reaction, and the antibody variants comprising each population are determined by next-generation sequencing.

In embodiments, the relative binding affinity of each functional mold molecule variant for a functional mimic molecule is determined by computing an enrichment score (as by the ordinary least squares regression). See, e.g., Rubin et al., “A statistical framework for analyzing deep mutational scanning data,” Genome Biology volume 18, Article number: 150 (2017 ). For example, such a deep mutational scanning statistical model generates error estimates for each measurement, capturing both sampling error and consistency between replicates.

In embodiments, the relative binding affinity of each functional mold molecule variant for a functional mimic molecule is determined by computing mean bin methods. See, e.g., Starr et al., “Deep Mutational Scanning of SARS-CoV-2 Receptor Binding Domain Reveals Constraints on Folding and ACE2 Binding,” Cell, Volume 182, Issue 5 (2020). For example, computing mean bin of a variant phenotype can reveal its binding affinity.

In embodiments, the interaction evaluation comprises evaluating binding affinity interactions, wherein the binding affinity interactions are measured by cells expressing functional mold molecules surface display constructs stained with multiple concentrations of functional mimic molecules prior to sorting. Apparent dissociation constants can then be determined by fitting, for each variant, a Hill function to the number of observations, by sequencing, per concentration. See, e.g., Adams et al., “Measuring the sequence-affinity landscape of antibodies with massively parallel titration curves,” eLIFE, 5:e23156 (2016).

In embodiments, the interaction evaluation comprises evaluating binding affinity interactions, wherein the binding affinity interactions are measured as a pool wherein functional mold molecule variants are conjugated to unique DNA barcodes and used to stain cells expressing functional mimic molecules. In embodiments, relative affinities are determined by barcode enrichments after single cell sequencing. See, e.g., Stoeckius et al., “Simultaneous epitope and transcriptome measurement in single cells,” Nature Methods, Volume 14, pp. 865-868 (2017).

In embodiments, the disclosure provides for comparing the interaction of the plurality of variant functional mold molecules with i) the model and ii) the one or more candidate functional mimic molecules, in order to identify the functional mimic molecule having the target profile.

In embodiments, the comparison comprises the correlation (e.g., Pearson's rho, Spearman's rho, or Kendall's tau) between vectorized fingerprints.

In embodiments, the comparison comprises a measure of distance (e.g., Euclidean distance or Manhattan distance) between vectorized fingerprints.

In embodiments, the comparison comprises a collection of vectorized functional mimic molecule fingerprints that are clustered (e.g., as by k means clustering or affinity propagation), optionally wherein functional mimic molecules co-clustering with the model are considered similar.

In embodiments, the comparison comprises clustering that is performed on a low dimensional embedding (e.g., principal components, independent components, t-distributed stochastic neighbor embedding, or uniform manifold approximation) of functional mimic molecule fingerprints.

In embodiments, the comparison comprises fingerprints comprised of variants that include multiple mutations collapsed to single-mutation relative affinities through a global epistasis model. See, e.g., Otwinowski et al., “Inferring the shape of global epistasis,” PNAS, 115 (32) E7550-E7558.

In embodiments, single mutant relative affinities can be compared using any of the approaches selected from the correlation between vectorized fingerprints; a measure of distance between vectorized fingerprints; a collection of vectorized functional mimic molecule fingerprints that are clustered; clustering that is performed on a low dimensional embedding of functional mimic molecule fingerprints; and fingerprints comprised of variants that include multiple mutations collapsed to single-mutation relative affinities through a global epistasis model.

Alternatively, in embodiments, single mutant relative affinities can be converted into probability distributions such that each possible combination of amino acid sequence position and mutation is assigned a value representative of the probability of sampling a mutation at that position after sorting a population in which every position-mutation combination was equally represented prior to sorting. See, e.g., Kowalsky et al., “Rapid Fine Conformational Epitope Mapping Using Comprehensive Mutagenesis and Deep Sequencing,” J. Biol. Chemistry, Volume 290, Issue 44 pp. 26457-26470 (2015). Probability distribution representations of fingerprints can be compared using measures of divergence such as earth mover's distance or Kullback-Leibler divergence.

In embodiments, similarity metrics are calculated in discrete or overlapping contiguous or dis-contiguous regions of the functional mold polypeptide or candidate functional mimic polypeptide amino acid sequence or structure and experimentally determined or predicted in silico, such that a functional mimic polypeptide is considered a partial mimic if it bears high similarity in a subset of regions.

### Pharmaceutical Compositions and Formulations

In embodiments, the present disclosure pertains to pharmaceutical compositions comprising the compositions, e.g. mimic molecules (functional mimic polymer), and a pharmaceutically acceptable carrier or excipient.

Any pharmaceutical compositions described herein can be administered to a patient as a component of a composition that comprises a pharmaceutically acceptable carrier or vehicle. Such compositions can optionally comprise a suitable amount of a pharmaceutically acceptable excipient so as to provide the form for proper administration.

In embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a patient. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present disclosure includes the described pharmaceutical compositions (and/or additional therapeutic agents) in various formulations. Any pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In embodiments, the composition is in the form of a capsule. In embodiments, the composition is in the form of a tablet. In embodiments, the pharmaceutical composition is formulated in the form of a soft-gel capsule. In embodiments, the pharmaceutical composition is formulated in the form of a gelatin capsule. In embodiments, the pharmaceutical composition is formulated as a liquid.

Where necessary, the present pharmaceutical compositions (and/or additional therapeutic agents) can also include a solubilizing agent. Also, the agents can be delivered with a suitable vehicle or delivery device as known in the art. Combination therapies outlined herein can be co-delivered in a single delivery vehicle or delivery device.

The formulations comprising the present pharmaceutical compositions (and/or additional therapeutic agents) of the present disclosure may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any pharmaceutical compositions (and/or additional therapeutic agents) described herein is formulated in accordance with routine procedures as a composition adapted for a mode of administration described herein.

Routes of administration include, for example: topical, oral, intradermal, transdermal, subcutaneous, intramuscular, intraperitoneal, intravenous, intranasal, epidural, sublingual, intranasal, intracerebral, intravaginal, rectal, or by inhalation. Administration can be local or systemic. In embodiments, the administering is by an intravenous route. The mode of administration can be left to the discretion of the practitioner, and depends in-part upon the site of the medical condition. In most instances, administration results in the release of any agent described herein onto or into the affected site.

In embodiments, the pharmaceutical compositions (and/or additional therapeutic agents) described herein are formulated in accordance with routine procedures as a composition adapted for administration. For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation.

Compositions for topical delivery can be in the form of a cream, gel, ointment, lotion, spray, aqueous or oily suspensions, powders, or emulsions, for example. Increased skin permeability and penetration may be achieved by non-invasive methods, for example, with the use of any nanocarriers combined with any pharmaceutical composition (and/or additional therapeutic agents) described herein. The skin can act as a reservoir and can be used to deliver the compositions (and/or additional therapeutic agents) described herein for more extended periods in a sustained manner.

Any pharmaceutical compositions (and/or additional therapeutic agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled-or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The disclosure thus provides single unit dosage forms suitable for oral administration such as, but not limited to, tablets, capsules, gelcaps, and caplets that are adapted for controlled-or sustained-release.

Controlled-or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used. Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Definitions

The following definitions are used in connection with the invention disclosed herein. Unless defined otherwise, all technical and scientific terms used herein have the same meaning as commonly understood to one of skill in the art to which this invention belongs.

As used herein, “a,” “an,” or “the” can mean one or more than one. Further, the term “about” when used in connection with a referenced numeric indication means the referenced numeric indication plus or minus up to 10% of that referenced numeric indication. For example, the language “about 50” covers the range of 45 to 55.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

This disclosure is further illustrated by the following non-limiting examples.

### EXAMPLES

Without wishing to be bound by theory, the process for identifying mimics is analogous to the manufacturing process of casting a replica. First, a surface to be mimicked (the model) and a molecule that binds to that surface (the mold) were identified. Then, molecules that bind to the mold (candidate mimics) were identified. Finally, detailed characterization of the mold-candidate interactions was performed in order to identify candidates that imitate the model.

### EXAMPLE 1: IDENTIFICATION AND CHARACTERIZATION OF ANTIBODIES THAT MIMIC THE EPITOPE OF A COMMERCIAL ANTIBODY ON AN ANTIGEN

The experiments of this example demonstrate the process of identifying mimics and discovering and characterizing antibodies that mimic the epitope of a commercial antibody on an antigen. In this example, the antigen's commercial antibody epitope was used as the model and the commercial antibody was used as the mold.

Biopanning and anti-drug antibody (ADA) repertoire mining were performed to identify a collection of anti-idiotypic antibodies that bind to the commercial antibody. Deep mutational scanning of the commercial antibody amino acid sequence was then used to characterize the effects of nearly all possible heavy chain mutations on binding to both the antigen and the anti-idiotypic antibodies. Finally, comparing the profiles of mutational effects between the antigen and anti-idiotypic antibodies allowed for identification of antibodies that bind to the commercial antibody in a manner that finely mimics the antigen.

### Identification of Candidate Mimics

Specifically, biopanning against the commercial antibody was performed using a naïve human scFv phage display library. After three rounds of panning, 192 individual colonies were isolated and monoclonal phage ELISA assays were performed to measure binding to both the commercial antibody and isotype-matched polyclonal IgG. 99 scFv antibodies were identified showing at least two-fold greater binding to the commercial antibody than polyclonal IgG. These clones were then sent for Sanger sequencing of the VH and VL, which identified 56 unique clones and 41 clonotypes.

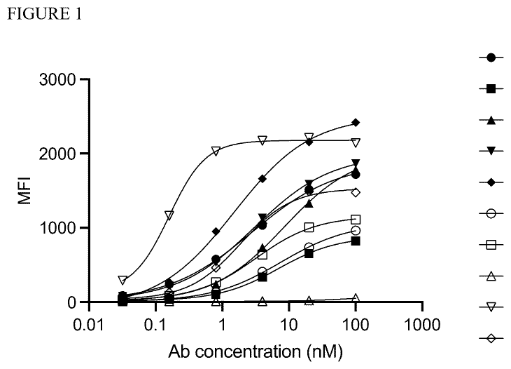

Use of Sanger sequences allowed for the determination of the heavy and light chain sequences of scFvs showing binding of the commercial antibody. Ten of these sequences were produced as IgG antibodies. Binding to yeast displayed commercial antibody Fab was measured by flow cytometry, as depicted in FIG. 1. EC50 values ranged from 0.2 nM to 8.5 nM, with one clone exhibiting minimal binding. See FIG. 1.

### Characterization of Mimicry

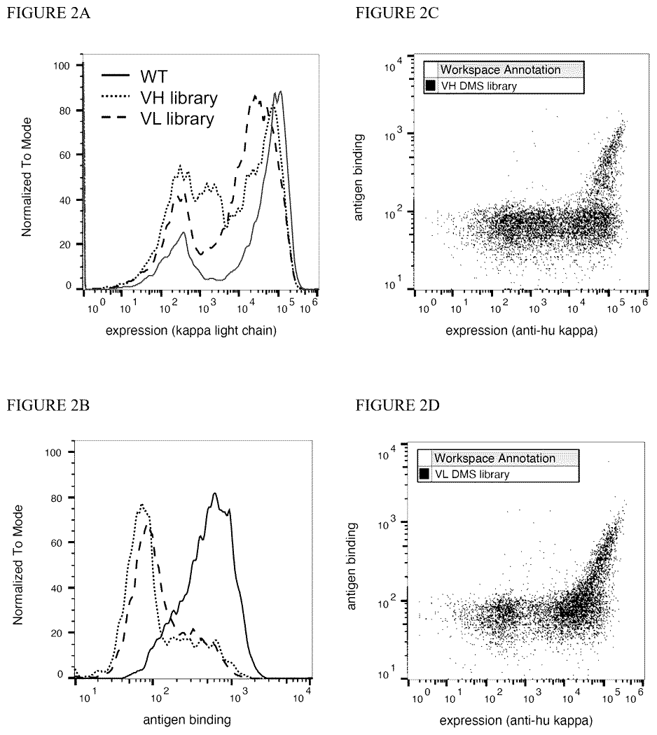

A yeast surface display deep mutational scanning variant DNA library of the heavy chain variable region of the commercial antibody was constructed by fragment mutagenesis. The library was then transformed into yeast and the library was validated for proper commercial antibody Fab surface display, measuring both surface expression and the antigen by flow cytometry. As depicted in FIGS. 2A-D, it was discovered that the antigen's binding was correlated with expression and that binding phenotypes were more variable (disrupted) within the yeast library than compared to reference type commercial antibody Fab.

To determine a reference binding profile representing the effects of mutations to the commercial antibody on the antigen's binding, selections were performed in which fluorescence activated cell sorting (FACS) was used to sort yeast transfected with the commercial antibody library into bins based on the degree to which they bound the antigen. The variants in each bin were then sequenced and a binding score for each variant was computed representing the extent to which its frequency was enriched by the antigen selection (FIGS. 2A-D). FIGS. 2A-D show commercial antibody WT and DMS library binding to the antigen. FIG. 2A depicts the presence of human kappa light chain confirms the surface expression of wildtype commercial antibody (solid) and commercial antibody libraries (dashed and dotted) on yeast. FIG. 2B shows that after gating for Fab expression, the libraries (dashed and dotted lines) exhibited a greater phenotypic range of binding to antigen relative to WT (solid). FIGS. 2C-D show that both libraries with mutations of either heavy chain or mutations in the light chain exhibit antigen binding correlated with expression, except for those variants that appear to lose binding altogether.

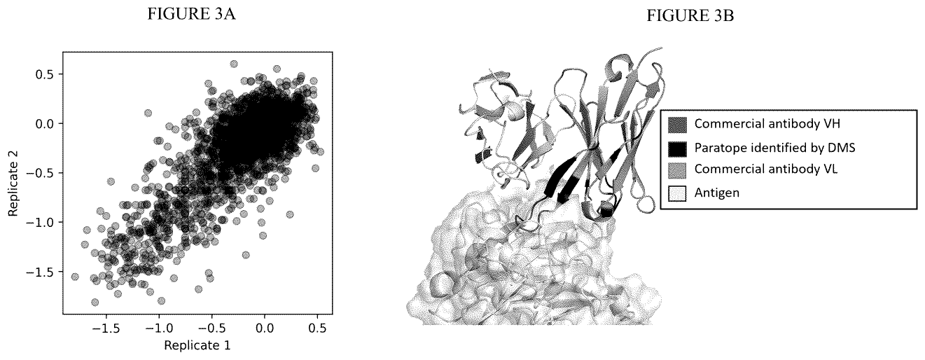

Selections were performed in duplicate and an inter-replicate replicate Pearson's correlation of 0.804 (FIG. 3A) was observed. To confirm that binding scores corresponded to the effects of mutations on the binding phenotype, an average score per sequence position was calculated. Regions with low average binding scores corresponded to interface residues in the commercial antibody/antigen co-crystal structure. FIG. 3B.

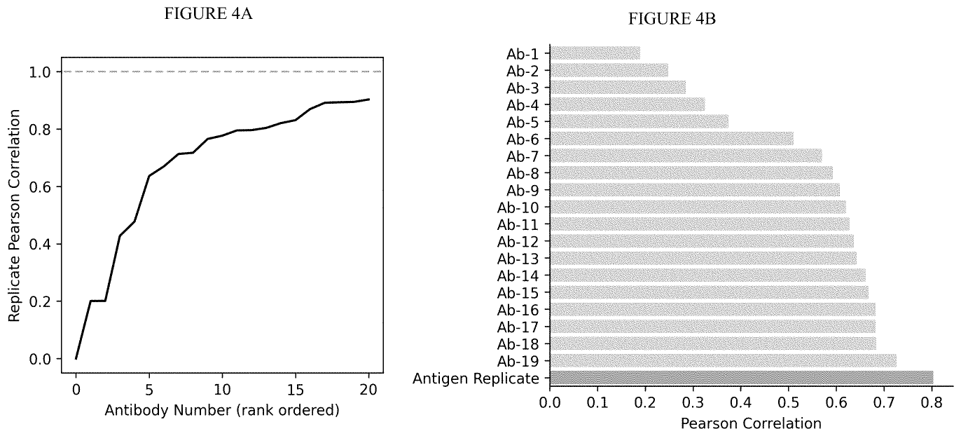

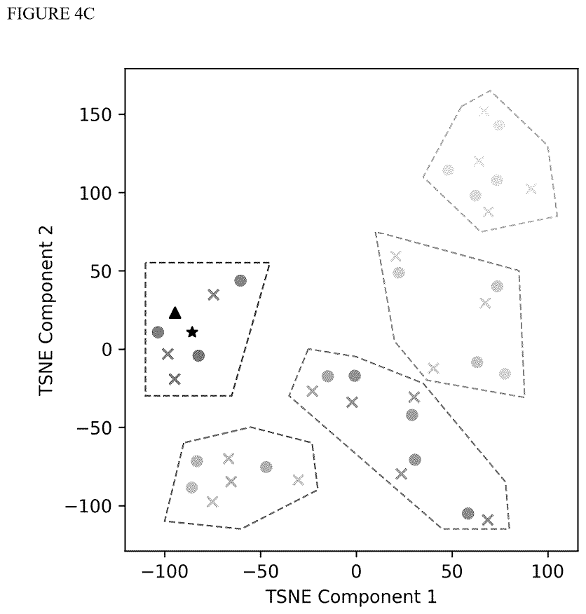

To characterize candidate mimics, additional selections were performed in which the commercial antibody library was sorted, in duplicate, into bins based on binding to one of 19 anti-idiotypic antibodies (nine identified by biopanning as described above and showing non-zero binding as IgG and ten derived from humans with anti-commercial antibody ADA responses). For each antibody, a binding score was calculated for each variant. Within antibody, inter-replicate binding scores were well correlated across variants across the majority of antibodies (FIG. 4A). To quantify mimicry, each antibody was compared to the antigen by calculating a mimicry score as the Pearson's correlation between binding score profiles across variants (FIG. 4B). Notably, the most positively correlated antibody displayed a mimicry score comparable to a replicate antigen selection.

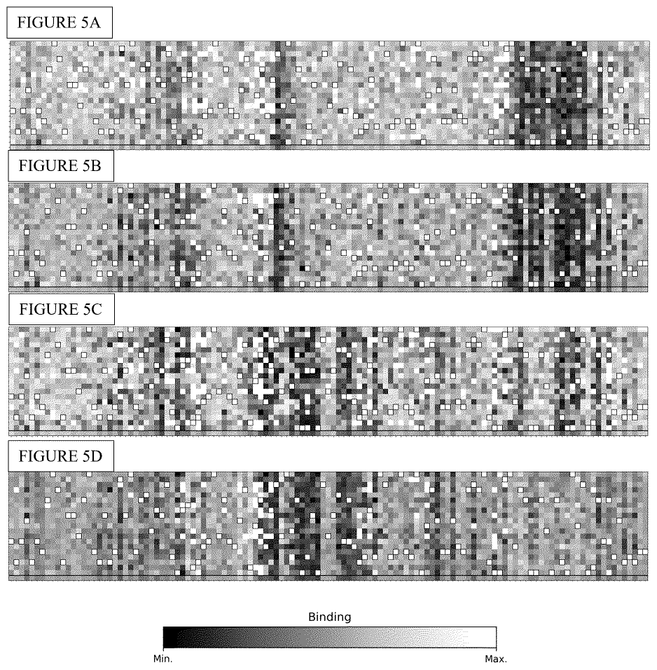

Next, clustering was performed to group the anti-idiotypic antibodies by their binding profiles across commercial antibody mutants. Clustering identified groups of binding profiles that mimic the antigen's, as well as more distant clusters (FIG. 4C). Further, exploration of representative binding profiles distinguished antibodies which bind the commercial antibody through distinct modes of interaction. For example, the interaction between an antibody that is specific to the commercial antibody but that shares few sensitive sites with the antigen was mapped (FIGS. 5A-B). It was also observed that an antibody that shares some but not all sensitive sites with the antigen seems to bind through a combination of CDRs 2 and 3 (FIG. 5C). Finally, an antibody was identified that had a nearly identical sensitivity profile to the antigen, as well as similar tolerances to the effects of individual mutations within-site (FIG. 5D).

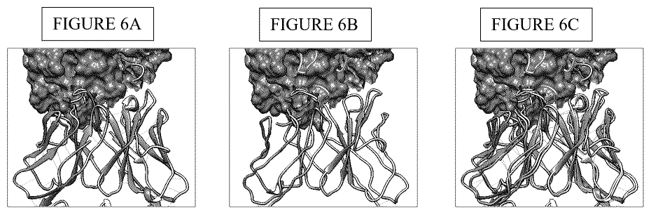

Cryogenic electron microscopy of anti-idiotypic antibodies in complex with the commercial antibody was performed to confirm similarities in binding. The complex structure of the same anti-commercial antibody displayed in FIG. 5B bound to the commercial antibody (FIG. 6A) shows a highly similar epitope footprint to a discovered and characterized mimic antibody (FIGS. 6B and 6C) despite minimal sequence similarity.

### EXAMPLE 2: IDENTIFICATION AND CHARACTERIZATION OF ANTIBODIES THAT MIMIC THE EPITOPE OF A KNOWN BROADLY NEUTRALIZING ANTIBODY

The experiments of this example demonstrate the process of identifying mimics and discovering and characterizing antibodies that mimic the epitope of a known broadly neutralizing antibody against an infectious-species antigen. In this example, the epitope of infectious species antigen was used as the model and the known broadly neutralizing antibody was used as the mold.

Biopanning against the broadly neutralizing antibody was used to identify an anti-idiotypic antibody. Deep mutational scanning of the broadly neutralizing antibody was used to characterize the effects on binding the discovered antibody, the model, and a known anti-idiotypic mimic antibody for nearly all possible heavy and light chain mutations. Characterization of the mutational profiles confirmed that the identified antibody mimics the antigen model. Mimicry was further validated by the experimentally determined structures of the complexes.

### Identification of Candidate Mimics

Biopanning against the broadly neutralizing antibody was performed using a naive human scFv phage display library. After three rounds of panning, 192 individual colonies were isolated and monoclonal phage ELISA assays were performed to measure binding to both the broadly neutralizing antibody and isotype-matched polyclonal IgG. Binding clones were then sent for Sanger sequencing of the VH and VL, and unique clones were produced as IgG antibodies.

### Characterization of Mimicry

A yeast surface display deep mutational scanning variant DNA library of the heavy chain variable region of the broadly neutralizing antibody was constructed and validated as described in Example 1, with the difference that antibody was in the single chain fragment variable (scFv) format.

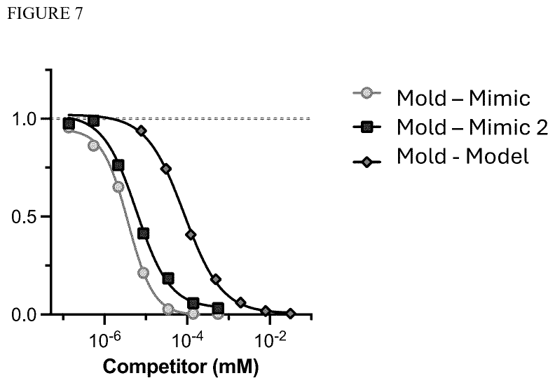

Magnetically activated cell sorting (MACS) was used to characterize the binding of individual members of the broadly neutralizing antibody library to both the known mimic antibody and the discovered antibody. Each antibody was bound to the library-expressing yeast population, and bound yeast were captured by magnetic bead. The bound and unbound populations were isolated, and the relative abundances of each variant in each population was measured by next generation sequencing. A binding score was calculated for each variant based on the relative enrichments in the bound and unbound populations. The library was also characterized for binding to the model antigen protein. Due to the lower affinity of broadly neutralizing antibody for the antigen protein (see FIG. 7, grey diamonds), FACS sorting was performed, as described in Example 1.

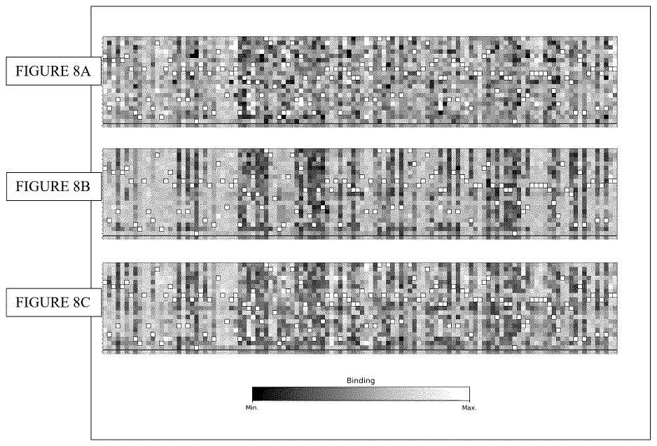

The binding profiles of the antibodies and antigen are depicted in FIGS. 8A-C. Comparison of the antigen protein (FIG. 8A) to the known mimic antibody profile (FIG. 8B) showed that key sensitive sites were conserved, with one notably different sensitive site in the middle of the CDR1 region. The discovered antibody (FIG. 8C), showed highly similar sensitive sites to the known mimic, but displayed a similar pattern to the model in the CDR1 region. Despite having a highly divergent sequence, the discovered antibody had not only similar sensitive sites, but also similar effects for individual mutations, indicating antigen mimicry.

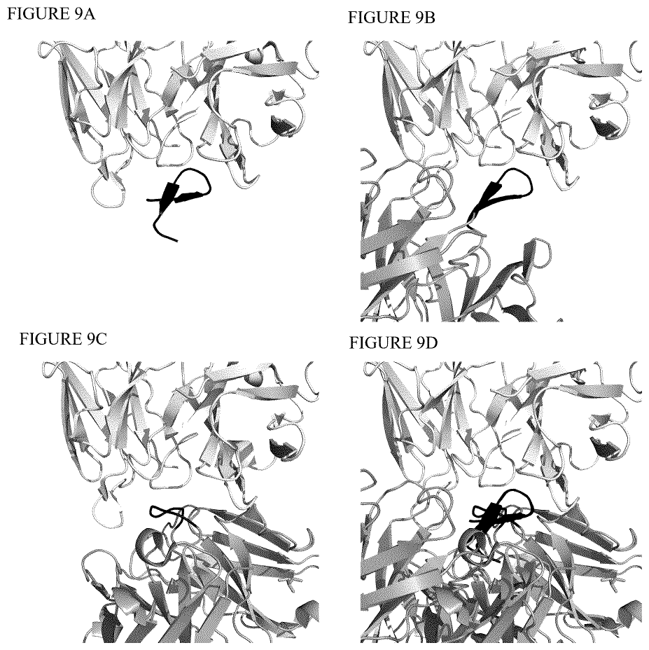

Cryogenic electron microscopy of the two mimic antibodies in complex with the broadly neutralizing antibody was performed, and derived structures were compared to the published structure of the broadly neutralizing antibody in complex with the model epitope peptide. The epitope forms a single loop structure that interacts with the heavy chain CDR1, heavy chain CDR3, and light chain CDR3 of the broadly neutralizing antibody (FIG. 9A). The heavy chain CDR3 of the discovered mimic formed a highly similar loop which binds in the same location (FIG. 9B). The discovered mimic antibody and known mimic antibody were observed to bind to the same region of the broadly neutralizing antibody, although with different docking angles (FIGS. 9C and 9D). Additionally, the structure of the loop mimicking the native epitope was different.

### EXAMPLE 3: IDENTIFICATION AND CHARACTERIZATION OF ANTIBODIES THAT MIMIC A NATIVE PROTEIN'S BINDING INTERACTION WITH A CELL SURFACE RECEPTOR

The experiments in this example demonstrate the process of identifying and characterizing mimics of a native protein (model) using its natural protein receptor as a mold.

Biopanning against a soluble form of the receptor (mold) was performed to identify a set of antibodies. In vitro characterization of candidate mimics was performed to confirm binding to the soluble mold and to confirm competitive binding with the model in both soluble and cell-surface presented forms of the mold. Deep mutational scanning of the sequence of the soluble extracellular domain of the mold was used to characterize the binding of candidate mimic antibodies, as well as the model protein and a known non-mimic antibody. Comparison of mutational effect profiles enabled identification of antibodies that compete and finely mimic the native protein.

### Identification of Mimics

Multiple rounds of biopanning were performed against a soluble form of the extracellular domain of the mold using a naive human scFv phage display library. After three rounds of selection, 192 individual colonies were isolated, sequenced by Sanger sequencing and characterized by phage ELISA for binding to the mold protein and polyclonal IgG. 36 unique scFv sequences were selected and converted to IgG for production.

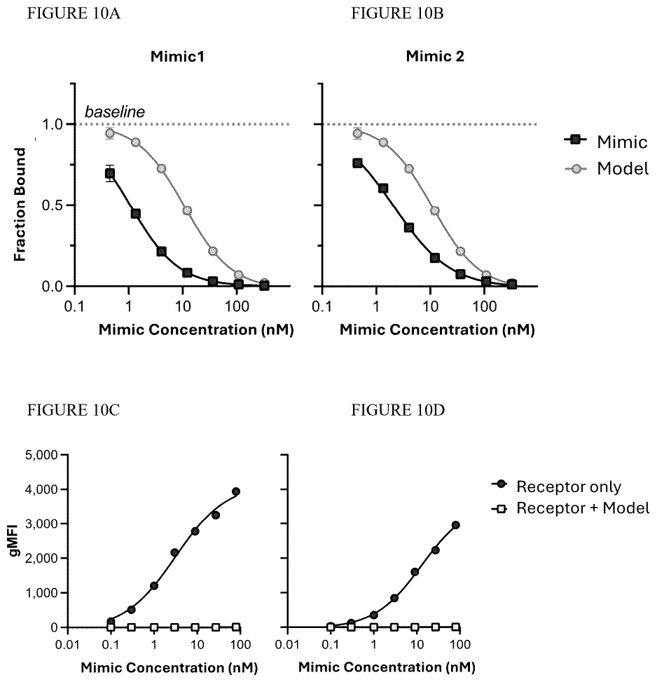

Candidate mimics were characterized for competitive binding with the model to the mold by ELISA. The extracellular domain of the mold receptor as a human Fc fusion was immobilized on the surface and bound to the model protein. Dissociation of the model from the mold was measured by chemiluminescence while titrating the candidate mimic antibodies or unlabeled model protein (FIGS. 10A and 10B). Two candidate mimics were identified as strong competitive binders. Competition was further confirmed in the context of the mold protein receptor presented on the cell surface. Candidate mimic antibodies were characterized for binding to both HEK-293 cells expressing the full mold protein sequence and HEK-293 cells co-expressing both the full mold protein and a surface bound form of the model protein. Binding was measured while titrating the antibodies (FIGS. 10C and 10D). Antibodies showed strong binding in the absence of co-expressing model protein, but binding was fully inhibited when the model was present.

### Characterization of mimicry

A yeast surface display deep mutational scanning variant DNA library of the extracellular domain of the mold receptor was constructed and validated, as described in Example 1.

The deep mutational scanning library was used to characterize the effects of mold mutations on binding to the model protein, a known non-mimic antibody and discovered candidate mimic antibodies. The yeast library was bound to protein and sorted into bins gated on binding by FACS, as described in Example 1.

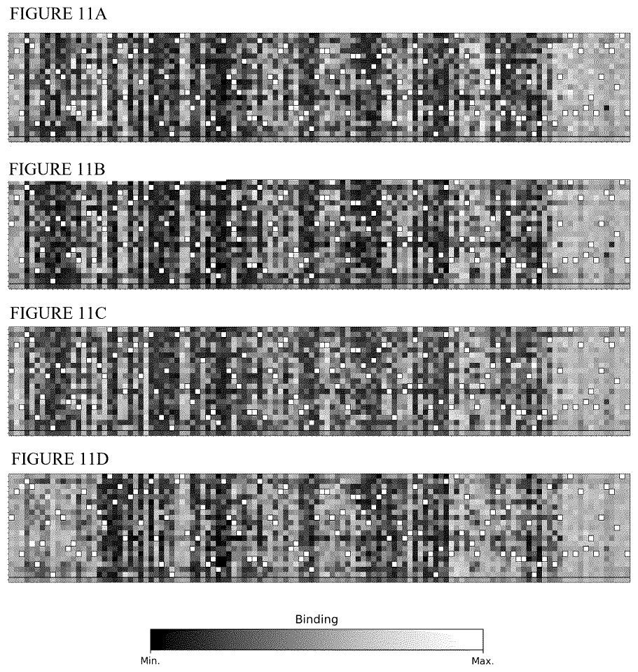

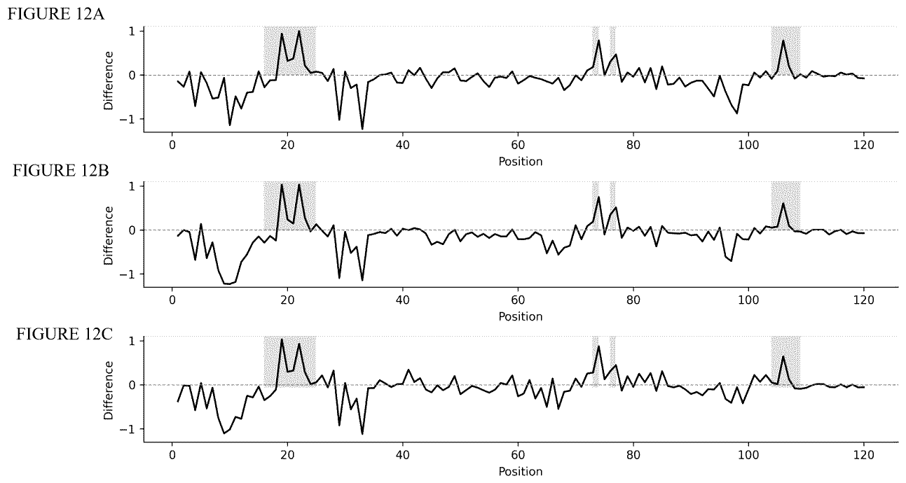

Two discovered antibodies appeared to be mimics: the binding profiles (FIGS. 11B and 11C) were similar to that of the model, as determined by the conservation of sensitive sites (FIG. 11A). The known non-mimic, in contrast, showed different sensitivities, particularly between residues 4 and 30 (FIG. 11D). A differential comparison of the mean position scores with the known non-mimic antibody showed an increase in binding signal for the model protein in the receptor mold residues observed to interact with model protein in a published crystal structure of the complex, highlighted in grey (FIG. 12A). Discovered mimic antibodies showed increases in the same sites and a nearly identical overall pattern (FIGS. 12B and 12C).

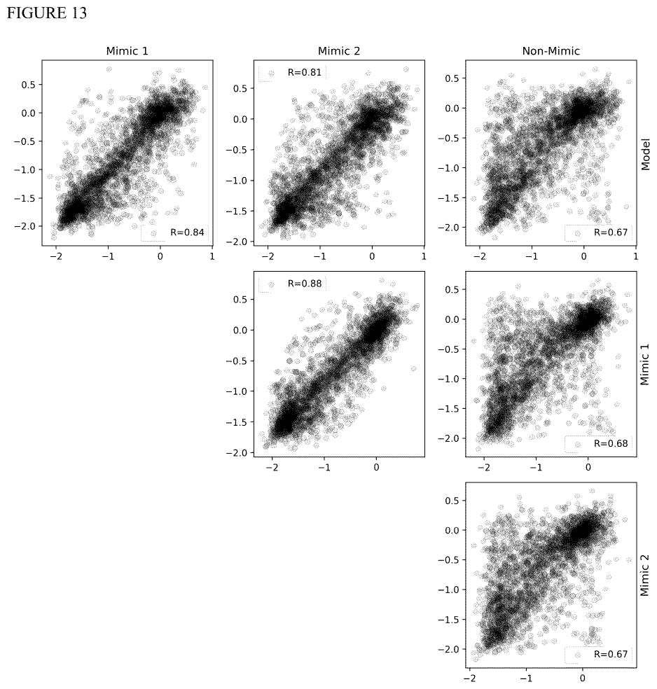

Individual mutation effect scores were highly correlated between the model protein and the discovered mimic antibodies with Pearson's correlation coefficients of 0.81 and 0.84 respectively (FIG. 13). Although divergent in sequence, the profiles of the two discovered antibody mimics were highly correlated, with a Pearson's correlation coefficient equal to 0.88. The known non-mimic antibody was found to be less correlated to both the model and mimic antibodies, with a Pearson's correlation coefficients ranging from 0.67 to 0.68 (FIG. 13).

Together, these data demonstrate that the described process for identifying mimics can discover and discriminate between antibodies with varying degrees of mimicry and to a variety of different model proteins.

### EQUIVALENTS

While the invention has been described in connection with embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method of identifying a functional mimic molecule having a target functional profile, the target functional profile comprising one or more functions of a model, the method comprising:
a) providing one or more variant libraries of one or more functional mold molecules of the model, wherein the variant library comprises a plurality of variants of the one or more functional mold molecules;
b) providing one or more candidate functional mimic molecules;
c) evaluating the interaction of the plurality of variant functional mold molecules with:
i) the model; and
ii) the one or more candidate functional mimic molecules, and comparing the evaluations of i) and ii);
d) identifying one or more functional mimic molecules having the target functional profile on the basis of the evaluation in c).

2. A method of identifying a functional mimic molecule having a target functional profile, the target functional profile comprising one or more functions of a model, the method comprising:
a) providing one or more functional mold molecules of the model;
b) providing one or more variant libraries of one or more candidate functional mimic molecules, the variant libraries comprising a plurality of variants of the one or more candidate functional mimic molecules;
c) evaluating the interaction of the one or more functional mold molecules with:
i) the model; and ii) the plurality of variants of the one or more candidate functional mimic molecules, and comparing the evaluations of i) and ii);
d) identifying one or more functional mimic molecules having the target functional profile on the basis of the evaluation in c).

3. A method of identifying a functional mimic molecule having a target functional profile, the target functional profile comprising one or more functions of a model, the method comprising:
a) providing one or more variant libraries of one or more functional mold molecules of the model, the variant libraries comprising a plurality of variants of the one or more functional mold molecules;
b) providing one or more variant libraries of one or more candidate functional mimic molecules, the variant libraries comprising a plurality of variants of the one or more candidate functional mimic molecules;
c) evaluating the interaction of the plurality of variant functional mold molecules with:
i) the model; and ii) the plurality of variant candidate functional mimic molecules, and comparing the evaluations of i) and ii);
d) identifying one or more functional mimic molecules having the target functional profile on the basis of the evaluation in c).

4. A method of identifying a functional mimic molecule having a target functional profile, the target functional profile comprising one or more functions of a model, the method comprising:
a) evaluating the interaction of a plurality of variant functional mold molecules with:
i) the model; and
ii) one or more candidate functional mimic molecules, and comparing the evaluations of i) and ii);
b) identifying one or more functional mimic molecules having the target functional profile on the basis of the evaluation in a).

5. The method of any one of claims 1-4, wherein the mimic molecule exhibits a non-zero similarity to the functional profile of the model.

6. The method of any one of the above claims, wherein the candidate functional mimic molecule is or comprises an immunoglobulin antibody, an antibody-like molecule, an antibody format, or an antigen-binding fragment thereof.

7. The method of claim 6, wherein the antibody or antibody-like molecule is selected from a bi-specific antibody, a tri-specific antibody, and an antibody-drug conjugate.

8. The method of claim 6 or 7, wherein the class or isotype of the immunoglobulin antibody, antibody-like molecule, antibody format, or antigen-binding fragment thereof, is selected from IgG, IgA, IgD, IgE, and IgM.

9. The method of any one of claim 8, wherein the subclass of the immunoglobulin antibody, antibody-like molecule, antibody format, or antigen-binding fragment thereof, is selected from IgG1, IgG2, IgG3, and IgG4, and IgA1 and IgA2.

10. The method of any one of claims 6-9, wherein the immunoglobulin antibody or antibody-like molecule is monoclonal or polyclonal.

11. The method of any one of claims 6-9, wherein the antigen-binding fragment is selected from a single-domain antibody (sdAb), a variable domain of heavy-chain antibodies (VHH), a single-chain antibody (scFv), a shark heavy-chain-only antibody (VNAR), a Fv, a Fab, a Fab′, a F(ab′)₂, and a microprotein (cysteine knot protein, knottin).

12. The method of any one of claims 6-11, wherein the functional mimic molecule is or comprises an anti-idiotypic antibody, or fragment thereof, that binds the antigen-binding domain of the functional mold polypeptide.

13. The method of claim 12, wherein the antigen-binding domain of the functional mold molecule is or comprises a paratope.

14. The method any one of the above claims, wherein the candidate functional mimic molecule is or comprises a small molecule.

15. The method of any one of the above claims, wherein the candidate functional mimic molecule is or comprises a polymer.

16. The method of any one of the above claims, wherein the polymer is or comprises a nucleic acid, optionally wherein the nucleic acid is or comprises DNA, RNA, or a combination thereof.

17. The method of claim 15 or 16, wherein the nucleic acid is selected from a DNA aptamer, a DNA optimer, a RNA aptamer, and a RNA optimer.

18. The method of claim 17, wherein the aptamer and/or optimer comprises one or more non-canonical and/or modified nucleotides.

19. The method of claim 18, wherein the aptamer and/or optimer comprises an altered backbone.

20. The method of claim 15, wherein the polymer is or comprises a nucleic acid and the nucleic acid is a riboswitch ribozyme.

21. The method of claim 15, wherein the polymer is or comprises a polypeptide.

22. The method of claim 21, where the polypeptide comprises one or more non-canonical amino acids.

23. The method of claim 22, wherein the non-canonical amino acids are L-isomers of canonical or non-canonical amino acids (L-amino acids).

24. The method of claim 22, wherein the non-canonical amino acids are D-isomers of canonical or non-canonical amino acids (D-amino acids).

25. The method of any one of claims 21-24, wherein the polypeptide is a binding agent.

26. The method of claim 25, wherein the binding agent is or comprises a non-immunoglobulin-based binding domain.

27. The method of claim 26, wherein the non-immunoglobulin-based binding domain is selected from an affimer, an affilin, an affitin, an affibody, an alphabody, an anticalin, an avimer, a DARPin, a fynomer, a gastrobody, a Kunitz domain, a monobody, a nanoCLAMP, a repebody, a pronectin, a centyrin, and an obody.

28. The method of claim 26, wherein the non-immunoglobulin-based binding domain is or comprises an enzyme.

29. The method of claim 26, wherein the non-immunoglobulin-based binding domain is or comprises a signaling protein.

30. The method of claim 29, wherein the signaling protein is selected from a cytokine, growth factor, or hormone.

31. The method of claim 26, wherein the non-immunoglobulin-based binding domain is or comprises a structural protein.

32. The method of claim 31, wherein the structural protein is or comprises a receptor, or a fragment thereof.

33. The method of claim 26, wherein the non-immunoglobulin-based binding domain is or comprises a ligand.

34. The method of claim 26, wherein the non-immunoglobulin-based binding domain is or comprises an extracellular domain.

35. The method of claim 26, wherein the non-immunoglobulin-based binding domain is or comprises a bacterial nucleic acid binding protein.

36. The method of claim 35, wherein the bacterial nucleic acid binding protein is selected from a bacteriophage MS2 protein and a bacteriophage lambda N protein.

37. The method of any one of claims 17-36, wherein the binding agent is a functional fragment.

38. The method of any one of claims 17-37, wherein the binding agent is bifunctional.

39. The method of claim 38, wherein the bifunctional binding agent is selected from a fusion protein or non-fusion protein conjugate.

40. The method of any one of claims 17-39, wherein the binding agent is conjugated to a carbohydrate (e.g., polysaccharide), a lipid, or a small molecule.

41. The method of any one of claims 17-40, wherein the binding agent is glycosylated, lapidated, or otherwise conjugated.

42. The method of any one of the above claims, wherein the target functional profile comprises one or more functions of the mimic molecule or complex, relative to the model.

43. The method of claim 42, wherein the function is selected from one or more of the affinity, avidity, activity, specificity, and/or selectivity for a receptor or ligand; agonism, antagonism, and/or inhibition; molecular structure or surface features; and biological activity.

44. The method of claim 43, wherein the molecular structure or surface features comprises arrangements of charge, polarity, and/or hydrophobicity.

45. The method of claim 43, wherein the biological activity comprises modulation of cellular signaling, communication, and/or pathogen interactions.

46. The method of claim 43, wherein the biological activity comprises modulation of transcription, translation, and/or post-translational modification.

47. The method of any one of the above claims, wherein the model is or comprises a protein, a peptide, a lipid, a carbohydrate, a nucleic acid, small molecule, or an inorganic molecule.

48. The method of claim 47, wherein the model is or comprises a polypeptide.

49. The method of claim 47, wherein the model is or comprises a small molecule or nucleic acid.

50. The method of any one of claims 47-49, wherein the model comprises an antigen.

51. The method of claim 50, wherein the antigen is selected from a viral protein, a bacterial protein, a protozoan protein, an Archean protein, a fungal protein, a mammalian protein, or an inorganic protein, a small molecule, a carbohydrate, a nucleic acid, a lipid, or a plastic.

52. The method of any one of claims 47-51, wherein the model is or comprises a therapeutic molecule.

53. The method of claim 52, wherein the therapeutic molecule is a therapeutic antibody or antigen-binding fragment thereof.

54. The method of claim 53, wherein the antigen-binding fragment of the antibody is the paratope of the antibody.

55. The method of any one of the above claims, wherein the variant library comprises variants across the entire mold molecule or functional mimic molecule or a subset of residue positions or moieties of the mold molecule or functional mimic molecule.

56. The method of claim 55, wherein the subset of residue positions is or comprises a contiguous domain or set.

57. The method of claim 55, wherein the subset of residue positions is or comprises a non-contiguous domain or set.

58. The method of any one of claims 55-57, wherein the variant library is systematically engineered.

59. The method of claim 58, where the systematic engineering is or comprises an alanine scan, a shotgun scan, and/or binomial mutagenesis.

60. The method of claim 58, wherein the systematic engineering is or comprises site-directed mutagenesis.

61. The method of any one of claims 55-57, wherein the variant library is randomly engineered.

62. The method of any one of claims 55-57, wherein the variant library is semi-systematically engineered by random mutation or random mutation of specific set of residue positions.

63. The method of any one of claims 55-57, wherein the variant library is selected from a fixed amino acid substitution library, a subset of possible substitutions library, or a full-substitution library.

64. The method of claim 63, wherein the fixed amino acid substitution library is an alanine library.

65. The method of claim 63, wherein the full substitution library comprises deletions and insertions.

66. The method of any one of claims 55-65, wherein the variant library comprises variation across substantially the entire reference polypeptide.

67. The method of any one of claims 55-65, wherein the variant library comprises variation across a subset of polypeptide residues.

68. The method of any one of claims 55-67, wherein the variant library comprises immunoglobulin variable domains selected from variable heavy, variable light, or variable heavy and variable light domains, or a fragment thereof.

69. The method of claim 68, wherein the fragment comprises one or more of a hCDR1, hCDR2, hCDR 3, 1CDR1, 1CDR 2, and 1CDR 3.

70. The method of claim 68 or 69, wherein the fragment comprises 3 or 6 CDRs.

71. The method of any one of claims 55-70, wherein the variant library comprises all possible single mutations and a distribution of about 2, about 3, about 4, or more multiple mutants.

72. The method of claim 55, wherein the variant library is a deep mutational scan (DMS) library.

73. The method of any one of claims 55-72, wherein the variant library covers at least about 5%, about 10%, about 15%, about 20%, about 25%, about 30%, about 35%, about 40%, about 45%, about 50%, about 55%, about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, or about 99% of the average length of the functional mold molecule or functional mimic molecule.

74. The method of any one of claims 55-73, wherein the average length of the functional mold molecule or functional mimic molecule is at least about 10, about 20, about 30, about 40, about 50, about 60, about 70, about 80, about 90, about 100, about 110, about 120, about 130, about 140, or about 150 monomers, optionally wherein the monomer is an amino acid or a nucleotide.

75. The method of any one of claims 55-74, wherein the variant library comprises at least about 100, about 200, about 400, about 500, about 1,000, about 2,500, about 5,000, about 10,000, about 25,000, about 30,000, about 40,000, about 50,000, or about 60,000 distinct sequences.

76. The method of any one of claims 55-75, wherein the variant library comprises at least about 80%, about 85, about 90%, about 95%, or about 100% single position substitutions to a specific residue, relative to a reference polymer.

77. The method of claim 76, wherein the residue is an alanine residue.

78. The method of any one of claims 55-77, wherein the variant library comprises at least about 80%, about 85, about 90%, about 95%, or about 100% single position substitutions, insertions, and/or deletions to any residue.

79. The method of claim 78, wherein the residue is a non-identity residue of the 20 canonical amino acids.

80. The method of claim 78 or 79, wherein the at least about 1, about 2, about 3, about 4, about 5, about 6, about 7, about 8, about 9, or about 10 residues are deleted or inserted.

81. The method of any one of claims 55-80, wherein the variant library comprises combinations of mutations, optionally n % of all double substitutions, m % of all triple substitutions, or approximately following a negative binomial distribution with shape parameters approximately described by probability ~1−0.4*n_sucesses_{-3/4}.

82. The method of any one of claims 55-81, wherein the providing of one or more variant libraries of one or more functional mold molecules or functional mimic molecules further comprises generation of the one or more functional mold molecules or functional mimic molecules by a method selected from interactions reported in the scientific literature, phage display, immunization with the model, obtaining model-binding B cells from a subject previously exposed or suspected of being exposed to the model, competitive-binding assays, depletion studies, and structure-based modeling.

83. The method of any one of claims 55-82, wherein the variant library is or comprises soluble proteins or cell surface displayed constructs or phage display.

84. The method of claim 83, wherein the variant library soluble proteins are full Ig or fragments thereof.

85. The method of claim 83, wherein the cell surface displayed constructs comprises a yeast cell, a mammalian cell, an immortalized cell, or an insect cell.

86. The method of claim 85, wherein the yeast cell is selected from Saccharomyces cerevisiae, Schizosaccharomyces pombe, Pichia pastoris, Hansenula polymorpha, Yarrowia lipolytica, and Kluyveromyces lactis.

87. The method of claim 85, wherein the mammalian cell is a human cell.

88. The method of claim 85, wherein the mammalian cell is selected from a Chinese hamster ovary (CHO) cell, a baby hamster kidney (BHK) cell, a human embryonic kidney (HEK293T) cell, a Vero cell, or a Spodoptera frugiperda 9 (Sf9) cell.

89. The method of claim 88, wherein the CHO cell is a CHO-K1, a CHO-DHB11, a CHO-DXB1, a CHO-S, or a CHO-DG44 cell.

90. The method of claim 88 or 89, wherein the CHO cell comprises or is selected from CHO-K1 (ATCC CCL-61) cells, SURE CHO-M cells (derivative of CHO-K1), or baby hamster kidney cells (BHK, ATCC CCL-10).

91. The method of claim 88, wherein the Vero cell comprises or is selected from a Vero cell, a Vero 76 cell, and a Vero E6 cell.

92. The method of claim 85, wherein the cell is or comprises a human cervical carcinoma cell (HELA, ATCC CCL-2), 293 (ATCC CRL-1573), 3T3 (ATCC CCL-163), or a monkey kidney CV1 line (ATCC CCL-70).

93. The method of any one of claims 55-92, wherein elements of the variant library are expressed individually or as a pool.

94. The method of any one of claims 55-92, wherein the functional mold molecule exhibits a non-zero functional complementarity to a model or functional mimic molecule.

95. The method of claim 94, wherein the functional mold molecule is or comprises an immunoglobulin antibody, an antibody-like molecule, or an antigen-binding fragment thereof.

96. The method of claim 95, wherein the antibody or antibody-like molecule is selected from a bi-specific antibody, a tri-specific antibody, and an antibody-drug conjugate.

97. The method of claim 95 or 96, wherein the class or isotype of the immunoglobulin antibody, antibody-like molecule, or antigen-binding fragment thereof, is selected from IgG, IgA, IgD, IgE, and IgM.

98. The method of any one of claim 97, wherein the subclass of the immunoglobulin antibody, antibody-like molecule, or antigen-binding fragment thereof, is selected from IgG1, IgG2, IgG3, and IgG4, and IgA1 and IgA2.

99. The method of any one of claims 95-98, wherein the immunoglobulin antibody or antibody-like molecule is monoclonal or polyclonal.

100. The method of any one of claims 95-98, wherein the antigen-binding fragment is selected from a single-domain antibody (sdAb), a variable domain of heavy-chain antibodies (VHH), a single-chain antibody (scFv), a shark heavy-chain-only antibody (VNAR), a Fv, a Fab, a Fab′, a F(ab′)₂, and a microprotein (cysteine knot protein, knottin).

101. The method of any one of claims 95-100, wherein the functional mimic polypeptide is or comprises an anti-idiotypic antibody, or fragment thereof, that binds the antigen-binding domain of the functional mold polypeptide.

102. The method of claim 101, wherein the antigen-binding domain of the functional mold polypeptide is or comprises a paratope.

103. The method of claim 94, wherein the functional mold molecule is or comprises a small molecule.

104. The method of claim 94, wherein the functional mold molecule is or comprises a polymer.

105. The method of claim 104, wherein the polymer is or comprises a nucleic acid, optionally wherein the nucleic acid is or comprises DNA, RNA, or a combination thereof.

106. The method of claim 104 or 105, wherein the nucleic acid is selected from a DNA aptamer, a DNA optimer, a RNA aptamer, and a RNA optimer.

107. The method of claim 106, wherein the aptamer and/or optimer comprises one or more non-canonical and/or modified nucleotides.

108. The method of claim 106, wherein the aptamer and/or optimer comprises an altered backbone.

109. The method of claim 104, wherein the polymer is or comprises a nucleic acid and the nucleic acid is a riboswitch ribozyme.

110. The method of claim 104, wherein the polymer is or comprises a polypeptide.

111. The method of claim 110, where the polypeptide comprises one or more non-canonical amino acids.

112. The method of claim 111, wherein the non-canonical amino acids are L-isomers of canonical or non-canonical amino acids (L-amino acids).

113. The method of claim 111, wherein the non-canonical amino acids are D-isomers of canonical or non-canonical amino acids (D-amino acids).

114. The method of any one of claims 110-113, wherein the polypeptide is a binding agent.

115. The method of claim 114, wherein the binding agent is or comprises a non-immunoglobulin-based binding domain.

116. The method of claim 115, wherein the non-immunoglobulin-based binding domain is selected from an affimer, an affilin, an affitin, an affibody, an alphabody, an anticalin, an avimer, a DARPin, a fynomer, a gastrobody, a Kunitz domain, a monobody, a nanoCLAMP, a repebody, a pronectin, a centyrin, and an obody.

117. The method of claim 115, wherein the non-immunoglobulin-based binding domain is or comprises an enzyme.

118. The method of claim 115, wherein the non-immunoglobulin-based binding domain is or comprises a signaling protein.

119. The method of claim 118, wherein the signaling protein is selected from a cytokine, growth factor, or hormone.

120. The method of claim 115, wherein the non-immunoglobulin-based binding domain is or comprises a structural protein.

121. The method of claim 120, wherein the structural protein is or comprises a receptor, or a fragment thereof.

122. The method of claim 115, wherein the non-immunoglobulin-based binding domain is or comprises a ligand.

123. The method of claim 115, wherein the non-immunoglobulin-based binding domain is or comprises an extracellular domain.

124. The method of claim 115, wherein the non-immunoglobulin-based binding domain is or comprises a bacterial nucleic acid binding protein.

125. The method of claim 124, wherein the bacterial nucleic acid binding protein is selected from a bacteriophage MS2 protein and a bacteriophage lambda N protein.

126. The method of any one of claims 115-125, wherein the binding agent is a functional fragment.

127. The method of any one of claims 115-126, wherein the binding agent is bifunctional.

128. The method of claim 127, wherein the bifunctional binding agent is selected from a fusion protein or non-fusion protein conjugate.

129. The method of any one of claims 115-128, wherein the binding agent is conjugated to a carbohydrate (e.g., polysaccharide), a lipid, or a small molecule.

130. The method of any one of claims 115-129, wherein the binding agent is glycosylated, lapidated, or otherwise conjugated.

131. The method of any one of the above claims, wherein the evaluation in c) further comprises calculating a metric of similarity between a first vector comprising a model fingerprint and a second vector comprising a functional mimic molecule fingerprint.

132. The method of any one of the above claims, wherein the interaction evaluation is of binding affinity of the variant functional mold molecules to i) the target and ii) the one or more functional mimic molecules.

133. The method of any one of the above claims, wherein the evaluation in c) further comprises evaluating binding affinity interactions.

134. The method of claim 133, wherein the binding affinity interactions are measured by one or more of a chemical assay, an optical assay, and a radioactive assay.

135. The method of claim 134, wherein the chemical assay is selected from one or more of gel electrophoresis, enzyme-linked immunosorbent assay (ELISA), and immunoblotting.

136. The method of claim 135, wherein the immunoblot is a western blot.

137. The method of claim 134, wherein the optical assay is selected from one or more of a fluorescence intensity assay, a fluorescent anisotropy assay, and a fluorescence resonance energy transfer (FRET) assay.

138. The method of claim 134, wherein the radioactive binding assay is a radioimmunoassay.

139. The method of claim 133, wherein the binding affinity interactions are measured individually by flow cytometry, biolayer interferometry, surface plasmon resonance, or enzyme-linked immunosorbent assay.

140. The method of any one of claims 131-138, wherein the binding affinity interactions are measured as a pool.

141. The method of claim 140, wherein the pooled measurement comprises conjugating a candidate functional mimic molecule to a fluorophore or magnetic bead staining cells expressing variants of functional mold molecules as surface display constructs.

142. The method of claim 141, wherein the pooled measurement further comprises separating bound and unbound populations of candidate functional mimic molecules by fluorescence-activated cell sorting or magnetic-activated cell sorting.

143. The method of claim 142, wherein the binding affinity interactions are measured by cells expressing functional mold molecules surface display constructs stained with multiple concentrations of functional mimic molecules prior to sorting.

144. The method of claim 143, wherein the pooled measurement further comprises conjugating a functional mold molecule variant to a unique DNA barcode and staining cells expressing functional mimic molecules, wherein relative affinities are determined by barcode enrichments after single-cell sequencing.

145. A composition comprising a mimic molecule identified by:
a) providing one or more variant libraries of one or more functional mold molecules of the model, wherein the variant library comprises a plurality of variants of the one or more functional mold molecules;
b) providing one or more candidate functional mimic molecules;
c) evaluating the interaction of the plurality of variant functional mold molecules with:
i) the model; and
ii) the one or more candidate functional mimic molecules, and comparing the evaluations of i) and ii);
d) identifying one or more functional mimic molecules having the target functional profile on the basis of the evaluation in c).

146. A composition comprising a mimic molecule identified by:
a) providing one or more functional mold molecules of the model;
b) providing one or more variant libraries of one or more candidate functional mimic molecules, the variant libraries comprising a plurality of variants of the one or more candidate functional mimic molecules;
c) evaluating the interaction of the one or more functional mold molecules with:
i) the model; and ii) the plurality of variants of the one or more candidate functional mimic molecules, and comparing the evaluations of i) and ii);
d) identifying one or more functional mimic molecules having the target functional profile on the basis of the evaluation in c).

147. A composition comprising a mimic molecule identified by:
a) providing one or more variant libraries of one or more functional mold molecules of the model, the variant libraries comprising a plurality of variants of the one or more functional mold molecules;
b) providing one or more variant libraries of one or more candidate functional mimic molecules, the variant libraries comprising a plurality of variants of the one or more candidate functional mimic molecules;
c) evaluating the interaction of the plurality of variant functional mold molecules with:
i) the model; and ii) the plurality of variant candidate functional mimic molecules, and comparing the evaluations of i) and ii);
d) identifying one or more functional mimic molecules having the target functional profile on the basis of the evaluation in c).

148. A composition comprising a mimic molecule identified by:
a) evaluating the interaction of a plurality of variant functional mold molecules with:
i) the model; and
ii) one or more candidate functional mimic molecules, and comparing the evaluations of i) and ii);
b) identifying one or more functional mimic molecules having the target functional profile on the basis of the evaluation in a).

149. A composition comprising a functional mimic molecule identified by the method of any one of claims 1-144.

150. A method of making a therapy comprising a mimic molecule, wherein the method comprises:
(a) identifying the mimic molecule by a method of any one of claims 1-4, and (b) formulating the mimic molecule for administration to a subject in need thereof.
